SEQUENCE LISTING (13 sequences)

SEQ ID NO: 1 - protein, 1361 aa, homo sapien
MGAAGRQDFLFKAMLTISWLTLTCFPGATSTVAAGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 2 - protein, 510 aa, homo sapien
MRPGLSFLLALLFFLGQAAGDLGDVGPPIPSPGFSSFPGVDSSSSFSSSSRSGSSSSRSLGSGGSVSQLFSNFTGSVDDRGTCQCSVSLPDTTFPVDRVERLEFTAHVLSQKFEKELSKVREYVQLISVYEKKLLNLTVRIDIMEKDTISYTELDFELIKVEVKEMEKLVIQLKESFGGSSEIVDQLEVEIRNMTLLVEKLETLDKNNVLAIRREIVALKTKLKECEASKDQNTPVVHPPPTPGSCGHGGVVNISKPSVVQLNWRGFSYLYGAWGRDYSPQHPNKGLYWVAPLNTDGRLLEYYRLYNTLDDLLLYINARELRITYGQGSGTAVYNNNMYVNMYNTGNIARVNLTTNTIAVTQTLPNAAYNNRFSYANVAWQDIDFAVDENGLWVIYSTEASTGNMVISKLNDTTLQVLNTWYTKQYKPSASNAFMVCGVLYATRTMNTRTEEIFYYYDTNTGKEGKLDIVMHKMQEKVQSINYNPFDQKLYVYNDGYLLNYDLSVLQKPQ

SEQ ID NO: 3 - protein, 411 aa, homo sapien
MWSGWWLWPLVAVCTADFFRDEAERIMRDSPVIDGHNDLPWQLLDMFNNRLQDERANLTTLAGTHTNIPKLRAGFVGGQFWSVYTPCDTQNKDAVRRTLEQMDVVHRMCRMYPETFLYVTSSAGIRQAFREGKVASLIGVEGGHSIDSSLGVLRALYQLGMRYLTLTHSCNTPWADNWLVDTGDSEPQSQGLSPFGQRVVKELNRLGVLIDLAHVSVATMKATLQLSRAPVIFSHSSAYSVCASRRNVPDDVLRLVKQTDSLVMVNFYNNYISCTNKANLSQVADHLDHIKEVAGARAVGFGGDFDGVPRVPEGLEDVSKYPDLIAELLRRNWTEAEVKGALADNLLRVFEAVEQASNLTQAPEEEPIPLDQLGGSCRTHYGYSSGASSLHRHWGLLLASLAPLVLCLSLL

SEQ ID NO: 4 - protein, 411 aa, homo sapien; identical to SEQ ID NO: 3

SEQ ID NO: 5 - protein, 105 aa, homo sapien
MAKISSPTETERCIESLIAVFQKYAGKDGYNYTLSKTEFLSFMNTELAAFTKNQKDPGVLDRMMKKLDTNSDGQLDFSEFLNLIGGLAMACHDSFLKAVPSQKRT

SEQ ID NO: 6 - protein, 1140 aa, homo sapien
RAHSAVASCPRGGAAGRRARPVPGVGPGAAARGGEGGCGSSSAAASDPARSAGRPLQVPAPLPVRPPMAAAGQLCLLYLSAGLLSRLGAAFNLDTREDNVIRKYGDPGSLFGFSLAMHWQLQPEDKRLLLVGAPRAEALPLQRANRTGGLYSCDITARGPCTRIEFDNDADPTSESKEDQWMGVTVQSQGPGGKVVTCAHRYEKRQHVNTKQESRDIFGRCYVLSQNLRIEDDMDGGDWSFCDGRLRGHEKFGSCQQGVAATFTKDFHYIVFGAPGTYNWKGIVRVEQKNNTFFDMNIFEDGPYEVGGETEHDESLVPVPANSYLGFSLDSGKGIVSKDEITFVSGAPRANHSGAVVLLKRDMKSAHLLPEHIFDGEGLASSFGYDVAVVDLNKDGWQDIVIGAPQYFDRDGEVGGAVYVYMNQQGRWNNVKPIRLNGTKDSMFGIAVKNIGDINQDGYPDIAVGAPYDDLGKVFIYHGSANGINTKPTQVLKGISPYFGYSIAGNMDLDRNSYPDVAVGSLSDSVTIFRSRPVINIQKTITVTPNRIDLRQKTACGAPSGICLQVKSCFEYTANPAGYNPSISIVGTLEAEKERRKSGLSSRVQFRNQGSEPKYTQELTLKRQKQKVCMEETLWLQDNIRDKLRPIPITASVEIQEPSSRRRVNSLPEVLPILNSDEPKTAHIDVHFLKEGCGDDNVCNSNLKLEYKFCTREGNQDKFSYLPIQKGVPELVLKDQKDIALEITVTNSPSNPRNPTKDGDDAHEAKLIATFPDTLTYSAYRELRAFPEKQLSCVANQNGSQADCELGNPFKRNSNVTFYLVLSTTEVTFDTPDLDINLKLETTSNQDNLAPITAKAKVVIELLLSVSGVAKPSQVYFGGTVVGEQAMKSEDEVGSLIEYEFRVINLGKPLTNLGTATLNIQWPKEISNGKWLLYLVKVESKGLEKVTCEPQKEINSLNLTESHNSRKKREITEKQIDDNRKFSLFAERKYQTLNCSVNVNCVNIRCPLRGLDSKASLILRSRLWNSTFLEEYSKLNYLDILMRAFIDVTAAAENIRLPNAGTQVRVTVFPSKTVAQYSGVPWWIILVAILAGILMLALLVFILWKCGFFKRNKKDHYDATYHKAEIHAQPSDKERLTSDA

SEQ ID NO: 7 - protein, 1091 aa, homo sapien
MAAAGQLCLLYLSAGLLSRLGAAFNLDTREDNVIRKYGDPGSLFGFSLAMHWQLQPEDKRLLLVGAPRAEALPLQRANRTGGLYSCDITARGPCTRIEFDNDADPTSESKEDQWMGVTVQSQGPGGKVVTCAHRYEKRQHVNTKQESRDIFGRCYVLSQNLRIEDDMDGGDWSFCDGRLRGHEKFGSCQQGVAATFTKDFHYIVFGAPGTYNWKGIVRVEQKNNTFFDMNIFEDGPYEVGGETEHDESLVPVPANSYLGFSLDSGKGIVSKDEITFVSGAPRANHSGAVVLLKRDMKSAHLLPEHIFDGEGLASSFGYDVAVVDLNKDGWQDIVIGAPQYFDRDGEVGGAVYVYMNQQGRWNNVKPIRLNGTKDSMFGIAVKNIGDINQDGYPDIAVGAPYDDLGKVFIYHGSANGINTKPTQVLKGISPYFGYSIAGNMDLDRNSYPDVAVGSLSDSVTIFRSRPVINIQKTITVTPNRIDLRQKTACGAPSGICLQVKSCFEYTANPAGYNPSISIVGTLEAEKERRKSGLSSRVQFRNQGSEPKYTQELTLKRQKQKVCMEETLWLQDNIRDKLRPIPITASVEIQEPSSRRRVNSLPEVLPILNSDEPKTAHIDVHFLKEGCGDDNVCNSNLKLEYKFCTREGNQDKFSYLPIQKGVPELVLKDQKDIALEITVTNSPSNPRNPTKDGDDAHEAKLIATFPDTLTYSAYRELRAFPEKQLSCVANQNGSQADCELGNPFKRNSNVTFYLVLSTTEVTFDTPDLDINLKLETTSNQDNLAPITAKAKVVIELLLSVSGVAKPSQVYFGGTVVGEQAMKSEDEVGSLIEYEFRVINLGKPLTNLGTATLNIQWPKEISNGKWLLYLVKVESKGLEKVTCEPQKEINSLNLTESHNSRKKREITEKQIDDNRKFSLFAERKYQTLNCSVNVNCVNIRCPLRGLDSKASLILRSRLWNSTFLEEYSKLNYLDILMRAFIDVTAAAENIRLPNAGTQVRVTVFPSKTVAQYSGVPWWIILVAILAGILMLALLVFILWKCGFFKRSRYDDSVPRYHAVRIRKEEREIKDEKYIDNLEKKQWITKWNENESYS

SEQ ID NO: 8 - protein, 268 aa, homo sapien
YMRPRSGPTRNPRLRAFAGVPTRGRTRGQSRRCAAEASAGPERDARPGAPAAGTMGAAHSASEEVRELEGKTGFSSDQIEQLHRRFKQLSGDQPTIRKENFNNVPDLELNPIRSKIVRAFFDNRNLRKGPSGLADEINFEDFLTIMSYFRPIDTTMDEEQVELSRKEKLRFLFHMYDSDSDGRITLEEYRNVVEELLSGNPHIEKESARSIADGAMMEAASVCMGQMEPDQVYEGITFEDFLKIWQGIDIETKMHVRFLNMETMALCH

SEQ ID NO: 9 - protein, 241 aa, homo sapien
YMRPRSGPTRNPRLRAFAGVPTRGRTRGQSRRCAAEASAGPERDARPGAPAAGTMGAAHSASEEVRELEGKTGFSSDQIEQLHRRFKQLSGDQPTIRNLRKGPSGLADEINFEDFLTIMSYFRPIDTTMDEEQVELSRKEKLRFLFHMYDSDSDGRITLEEYRNVVEELLSGNPHIEKESARSIADGAMMEAASVCMGQMEPDQVYEGITFEDFLKIWQGIDIETKMHVRFLNMETMALCH

SEQ ID NO: 10 - protein, 158 aa, homo sapien
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 11 - protein, 158 aa, homo sapien; identical to SEQ ID NO: 10

SEQ ID NO: 12 - protein, 134 aa, homo sapien
MASRSMRLLLLLSCLAKTGVLGDIIMRPSCAPGWFYHKSNCYGYFRKLRNWSDAEVRNLLPAWPGLSRAKDQPEPQISFDSGSSVLPGHYEEKPLWLVKWREEGCVFNSFNSVSIAEAGAVCQTLDGLQAHTDT

SEQ ID NO: 13 - protein, 1212 aa, homo sapien
MEPRPTAPSSGAPGLAGVGETPSAAALAAARVELPGTAVPSVPEDAAPASRDGGGVRDEGPAAAGDGLGRPLGPTPSQSRFQVDLVSENAGRAAAAAAAAAAAAAAAGAGAGAKQTPADGEASGESEPAKGSEEAKGRFRVNFVDPAASSSAEDSLSDAAGVGVDGPNVSFQNGGDTVLSEGSSLHSGGGGGSGHHQHYYYDTHTNTYYLRTFGHNTMDAVPRIDHYRHTAAQLGEKLLRPSLAELHDELEKEPFEDGFANGEESTPTRDAVVTYTAESKGVVKFGWIKGVLVRCMLNIWGVMLFIRLSWIVGQAGIGLSVLVIMMATVVTTITGLSTSAIATNGFVRGGGAYYLISRSLGPEFGGAIGLIFAFANAVAVAMYVVGFAETVVELLKEHSILMIDEINDIRIIGAITVVILLGISVAGMEWEAKAQIVLLVILLLAIGDFVIGTFIPLESKKPKGFFGYKSEIFNENFGPDFREEETFFSVFAIFFPAATGILAGANISGDLADPQSAIPKGTLLAILITTLVYVGIAVSVGSCVVRDATGNVNDTIVTELTNCTSAACKLNFDFSSCESSPCSYGLMNNFQVMSMVSGFTPLISAGIFSATLSSALASLVSAPKIFQALCKDNIYPAFQMFAKGYGKNNEPLRGYILTFLIALGFILIAELNVIAPIISNFFLASYALINFSVFHASLAKSPGWRPAFKYYNMWISLLGAILCCIVMFVINWWAALLTYVIVLGLYIYVTYKKPDVNWGSSTQALTYLNALQHSIRLSGVEDHVKNFRPQCLVMTGAPNSRPALLHLVHDFTKNVGLMICGHVHMGPRRQAMKEMSIDQAKYQRWLIKNKMKAFYAPVHADDLREGAQYLMQAAGLGRMKPNTLVLGFKKDWLQADMRDVDMYINLFHDAFDIQYGVVVIRLKEGLDISHLQGQEELLSSQEKSPGTKDVVVSVEYSKKSDLDTSKPLSEKPITHKVEEEDGKTATQPLLKKESKGPIVPLNVADQKLLEASTQFQKKQGKNTIDVWWLFDDGGLTLLIPYLLTTKKKWKDCKIRVFIGGKINRIDHDRRAMATLLSKFRIDFSDIMVLGDINTKPKKENIIAFEEIIEPYRLHEDDKEQDIADKMKEDEPWRITDNELELYKTKTYRQIRLNELLKEHSSTANIIVMSLPVARKGAVSSALYMAWLEALSKDLPPILLVRGNHQSVLTFYS

(19) United States
(12) Patent Application Publication
(10) Pub. No.: US 2014/0155280 A1
(43) Pub. Date: Jun. 5, 2014

(54) METHOD OF DIAGNOSING NEOPLASMS

(71) Applicants: Lawrence Charles Lapointe, West Pennant Hills (AU); Susanne Kartin Pedersen, North Ryde (AU); Peter Molloy, Chatswood (AU)

(72) Inventors: Lawrence Charles Lapointe, West Pennant Hills (AU); Susanne Kartin Pedersen, North Ryde (AU); Peter Molloy, Chatswood (AU)

(73) Assignees: Commonwealth Scientific and Industrial Research Organisation, Campbell (AU); Clinical Genomics Pty. Ltd., North Ryde (AU)

(21) Appl. No.: 14/115,698

(22) PCT Filed: May 4, 2012
(86) PCT No.: PCT/AU2012/000483

§ 371 Date: Jan. 27, 2014

Related U.S. Application Data
(60) Provisional application No. 61/483,028, filed on May 5, 2011.

(51) Int. Cl. C12Q 1/68

(52) U.S. Cl. CPC C12Q 1/6886
USPC 506/7; 435/6.12; 435/7.92; 435/7.1

(57) ABSTRACT

The present invention relates generally to a method for screening a subject for the onset, predisposition to the onset and/or progression of a colorectal neoplasm by screening for modulation in the level of expression of one or more nucleic acid markers. More particularly, the present invention provides a method for screening a subject for the onset, predisposition to the onset and/or progression of a colorectal neoplasm by screening for modulation in the level of expression of one or more gene markers in membranous microvesicles. The expression profiles of the present invention are useful in a range of applications including, but not limited to, those relating to the diagnosing and/or monitoring of colorectal neoplasms, such as colorectal adenoma and adenocarcinomas.

### FIELD OF THE INVENTION

The present invention relates generally to a method for screening a subject for the onset, predisposition to the onset and/or progression of a colorectal neoplasm by screening for modulation in the level of expression of one or more nucleic acid markers. More particularly, the present invention provides a method for screening a subject for the onset, predisposition to the onset and/or progression of a colorectal neoplasm by screening for modulation in the level of expression of one or more gene markers in membranous microvesicles. The expression profiles of the present invention are useful in a range of applications including, but not limited to, those relating to the diagnosing and/or monitoring of colorectal neoplasms, such as colorectal adenoma and adenocarcinomas.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Adenomas are benign tumours, or neoplasms, of epithelial origin which are derived from glandular tissue or exhibit clearly defined glandular structures. Some adenomas show recognisable tissue elements, such as fibrous tissue (fibro adenomas) and epithelial structure, while others, such as bronchial adenomas, produce active compounds that might give rise to clinical syndromes.

Adenomas may progress to become an invasive neoplasm and are then termed adenocarcinomas. Accordingly, adenocarcinomas are defined as malignant epithelial tumours arising from glandular structures, which are constituent parts of many organs of the body. The term adenocarcinoma is also applied to tumours showing a glandular growth pattern. These tumours may be sub-classified according to the substances that they produce, for example mucus secreting and serous adenocarcinomas, or to the microscopic arrangement of their cells into patterns, for example papillary and follicular adenocarcinomas. These carcinomas may be solid or cystic (cystadenocarcinomas). Each organ may produce tumours showing a variety of histological types, for example the ovary may produce both mucinous and cystadenocarcinoma.

Adenomas in different organs behave differently. In general, the overall chance of carcinoma being present within an adenoma (i.e. a focus of cancer having developed within a benign lesion) is approximately 5%. However, this is related to size of an adenoma. For instance, in the large bowel (colon and rectum specifically) occurrence of a cancer within an adenoma is rare in adenomas of less than 1 centimetre. Such a development is estimated at 40 to 50% in adenomas which are greater than 4 centimetres and show certain histopathological change such as villous change, or high grade dysplasia. Adenomas with higher degrees of dysplasia have a higher incidence of carcinoma. In any given colorectal adenoma, the predictors of the presence of cancer now or the future occurrence of cancer in the organ include size (especially greater than 9 mm) degree of change from tubular to villous morphology, presence of high grade dysplasia and the morphological change described as “serrated adenoma”. In any given individual, the additional features of increasing age, familial occurrence of colorectal adenoma or cancer, male gender or multiplicity of adenomas, predict a future increased risk for cancer in the organ—so-called risk factors for cancer. Except for the presence of adenomas and its size, none of these is objectively defined and all those other than number and size are subject to observer error and to confusion as to precise definition of the feature in question. Because such factors can be difficult to assess and define, their value as predictors of current or future risk for cancer is imprecise.

Once a sporadic adenoma has developed, the chance of a new adenoma occurring is approximately 30% within 26 months.

Colorectal adenomas represent a class of adenomas which are exhibiting an increasing incidence, particularly in more affluent countries. The causes of adenoma, and of progression to adenocarcinoma, are still the subject of intensive research. To date it has been speculated that in addition to genetic predisposition, environmental factors (such as diet) play a role in the development of this condition. Most studies indicate that the relevant environmental factors relate to high dietary fat, low fibre, low vegetable intake, smoking, obesity, physical inactivity and high refined carbohydrates.

Colonic adenomas are localised areas of dysplastic epithelium which initially involve just one or several crypts and may not protrude from the surface, but with increased growth in size, usually resulting from an imbalance in proliferation and/or apoptosis, they may protrude. Adenomas can be classified in several ways. One is by their gross appearance and the major descriptors include degrees of protrusion: flat sessile (i.e. protruding but without a distinct stalk) or pedunculated (i.e. having a stalk). Other gross descriptors include actual size in the largest dimension and actual number in the colon/rectum. While small adenomas (less than say 5 or 10 millimetres) exhibit a smooth tan surface, pedunculated and especially larger adenomas tend to have a cobblestone or lobulated red-brown surface. Larger sessile adenomas may exhibit a more delicate villous surface. Another set of descriptors include the histopathological classification; the prime descriptors of clinical value include degree of dysplasia (low or high), whether or not a focus of invasive cancer is present, degree of change from tubular gland formation to villous gland formation (hence classification is tubular, villous or tubulovillous), presence of admixed hyperplastic change and of so-called “serrated” adenomas and its subgroups. Adenomas can be situated at any site in the colon and/or rectum although they tend to be more common in the rectum and distal colon. All of these descriptors, with the exception of number and size, are relatively subjective and subject to interobserver disagreement.

The various descriptive features of adenomas are of value not just to ascertain the neoplastic status of any given adenomas when detected, but also to predict a person's future risk of developing colorectal adenomas or cancer. Those features of an adenoma or number of adenomas in an individual that point to an increased future risk for cancer or recurrence of new adenomas include: size of the largest adenoma (especially 10 mm or larger), degree of villous change (especially at least 25% such change and particularly 100% such change), high grade dysplasia, number (3 or more of any size or histological status) or presence of serrated adenoma features. None except size or number is objective and all are relatively subjective and subject to interobserver disagreement. These predictors of risk for future neoplasia (hence “risk”) are vital in practice because they are used to determine the rate and need for and frequency of future colonoscopic surveillance. More accurate risk classification might thus reduce workload of colonoscopy, make it more cost-effective and reduce the risk of complications from unnecessary procedures.

Adenomas are generally asymptomatic, therefore rendering difficult their diagnosis and treatment at a stage prior to when they might develop invasive characteristics and so became cancer. It is technically impossible to predict the presence or absence of carcinoma based on the gross appearance of adenomas, although larger adenomas are more likely to show a region of malignant change than are smaller adenomas. Sessile adenomas exhibit a higher incidence of malignancy than pedunculated adenomas of the same size. Some adenomas result in blood loss which might be observed or detectable in the stools; while sometimes visible by eye, it is often, when it occurs, microscopic or “occult”. Larger adenomas tend to bleed more than smaller adenomas. However, since blood in the stool, whether overt or occult, can also be indicative of non-adenomatous conditions, the accurate diagnosis of adenoma is rendered difficult without the application of highly invasive procedures such as colonoscopy combined with tissue acquisition by either removal (i.e. polypectomy) or biopsy and subsequent histopathological analysis.

Accordingly, there is an on-going need to elucidate the causes of adenoma and to develop more informative diagnostic protocols or aids to diagnosis that enable one to direct colonoscopy at people more likely to have adenomas. These adenomas may be high risk, advanced or neither of these. Furthermore, it can be difficult after colonoscopy, to be certain that all adenomas have been removed, especially in a person who has had multiple adenomas. An accurate screening test may minimise the need to undertake an early second colonoscopy to ensure that the colon has been cleared of neoplasms. Accordingly, the identification of molecular markers for adenomas would provide means for understanding the cause of adenomas and cancer, improving diagnosis of adenomas including development of useful screening tests, elucidating the histological stage of an adenoma, characterising a patient's future risk for colorectal neoplasia on the basis of the molecular state of an adenoma and facilitating treatment of adenomas.

To date, research has tended to focus on the identification of gene mutations which are determinative of colorectal neoplasms. However, more recent findings have indicated that in fact that changes in the level of expression of unmutated genes which are expressed in healthy individuals are also indicative of neoplasm development. These gene expression changes are usually routinely detectable in colorectal tissue samples. However, from the patient perspective, harvesting colorectal tissue samples is invasive and not without risk in terms of post-operative complications, such as infection. The sampling of peripheral blood is generally a significantly preferred method in terms of harvesting a biological sample for analysis but is entirely dependent on whether or not the change in expression levels of the gene in issue are detectable in blood at levels which are diagnostically useful.

In work leading up to the present invention it has been determined that in the context of colorectal neoplasia gene markers, the level of expression of which are increased in neoplasia, this increase in expression is not necessarily easily detectable in the plasma due to issues of diagnostic sensitivity. However, it has been unexpectedly found that a small subset of these gene markers exhibit a significant increase in the level of their expression within circulating exosomes. This finding is unexpected since not all colorectal gene markers which are increased in expression in cancerous tissues were necessarily also increased in exosomes. This determination is particularly important since exosomes circulate in the periphery and can therefore be harvested easily, such as via a blood sample. Still further, whereas analysis of gene expression levels within whole blood or plasma can suffer from problems of lack of sensitivity, the determination that a small subgroup of previously identified colorectal neoplasia markers are actually detectable within the defined environment of an exosome now provides a significantly more sensitive detection means, due to the reduced levels of irrelevant contaminating genetic material. It is also highly desirable from the point of view that it involves the use of minimally invasive harvesting techniques, such as withdrawal of a blood sample.

### SUMMARY OF THE INVENTION

One aspect of the present invention is directed to a method of screening for the onset or predisposition to the onset of a large intestine neoplasm in an individual, said method comprising measuring the level of expression of:

(i) any one or more genes selected from:

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 |
| 9. S100A11 | 10. ACAA2 | 11. ANPEP | 12. ANXA3 |
| 13. APP | 14. APPL2 | 15. AZGP1 | 16. BGN |
| 17. c20orf199 | 18. CALR | 19. CAP1 | 20. COL12A1 |
| 21. CSE1L | 22. CTSC | 23. CXCL3 | 24. DMBT1 |
| 25. ENO1 | 26. EPS8L3 | 27. FAT | 28. FTH1 |
| 29. GALNT6 | 30. GMDS | 31. GNB2L1 | 32. GPRC5A |
| 33. HEPH | 34. HLADRB1 | 35. HPGD | 36. HSP9OAA1 |
| 37. IFITM1 | 38. IFITM2 | 39. KRT8 | 40. LCN2 |
| 41. LDHB | 42. LIMA1 | 43. LOC440264 | 44. LRPPRC |
| 45. LRSAM1 | 46. MLLT3 | 47. MMP7 | 48. MUC13 |
| 49. MYO5B | 50. NDRG1 | 51. NEBL | 52. NQO1 |
| 53. OLA1 | 54. PIGR | 55. PRDX1 | 56. PROS1 |
| 57. PSAT1 | 58. PUS7 | 59. RAB8A | 60. RPL6 |
| 61. RPS4X | 62. RPS7 | 63. S100A1 | 64. S100A6 |
| 65. S100P | 66. SLC39A5 | 67. SLC7A5 | 68. SLK |
| 69. SOD1 | 70. SORD | 71. TACSTD2 | 72. TCP1 |
| 73. TFRC | 74. TGFBI | 75. THBS2 | 76. TM7SF3 |
| 77. TUBB6 | 78. VAMP3 | 79. VAT1; | |

or
(ii) any one or more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | 4. chr16: 89,687,000-89,704,839 |
| 5. chr12: 117,476,728-117,537, | 6. chr5: 127,419,483-127,525,380 |
| 251 | |
| 7. chr2: 173,292,314-173,371,181 | 8. chr1: 120,336,641-120,354,203 |
| 9. chr1: 152,004,982-152,009,511 | 10. chr18: 47,309,874-47,340,251 |
| 11. chr15: 90,328,126-90,358,072 | 12. chr4: 79,472,742-79,531,605 |
| 13. chr21: 27,252,861-27,543,138 | 14. chr12: 105,567,075-105,630,008 |
| 15. chr7: 99,564,350-99,573,735 | 16. chrX: 152,760,347-152,775,004 |
| 17. chr20: 47894715-47905797 | 18. chr20: 47894715-47905797 |
| 19. chr20: 47894715-47905797 | 20. chr20: 47894715-47905797 |
| 21. chr20: 47894808-47905797 | 22. chr20: 47895179-47905797 |
| 23. chr20: 47895179-47905797 | 24. chr19: 13,049,414-13,055,304 |
| 25. chr1: 40,506,255-40,538,321 | 26. chr6: 75,794,042-75,915,623 |
| 27. chr20: 47,662,838-47,713,486 | 28. chr11: 88,026,760-88,070,941 |
| 29. chr4: 74,902,312-74,904,490 | 30. chr10: 124,320,181-124,403,252 |
| 31. chr1: 8,921,059-8,939,151 | 32. chr1: 110,292,702-110,306,644 |
| 33. chr4: 126315091-126414087 | 34. chr5: 150935821-150948505 |
| 35. chr4: 187627717-187647850 | 36. chr4: 187508937-187516980 |
| 37. chr5: 150883653-150948505 | 38. chr5: 150883653-150911531 |
| 39. chr4: 187508937-187644987 | 40. chr4: 126237567-126414087 |
| 41. chr4: 126369616-126412943 | 42. chr11: 92085262-92629635 |
| 43. chr11: 92573728-92629635 | 44. chr11: 61,731,757-61,735,132 |
| 45. chr12: 51,745,833-51,785,200 | 46. chr6: 1,624,035-2,245,868 |
| 47. chr5: 180,663,928-180,670,906 | 48. chr12: 13,043 956-13,066,600 |
| 49. chrX: 65,382,433-65,487,230 | 50. chr6: 3,774,425-3,787,546 |
| 51. chr4: 175,411,328-175,443,792 | 52. chr14: 102547075-102606086 |
| 53. chr11: 313,991-315,272 | 54. chr11: 308,107-309,410 |
| 55. chr12: 53,290,971-53,298,868 | 56. chr9: 130,911,732-130,915,734 |
| 57. chr12: 21,788,275-21,810,789 | 58. chr12: 50,569,563-50,677,353 |
| 59. chr15: 30,262,050-30,265,947 | 60. chr2: 44,113,363-44,223,144 |
| 61. chr9: 130,214,534-130,265,780 | 62. chr9: 20,344,968-20,622,514 |
| 63. chr11: 102,391,239-102,401, | 64. chr3: 124,624,289-124,653,595 |
| 478 | |
| 65. chr18: 47,349,156-47,721,451 | 66. chr8: 134,249,414-134,309,547 |
| 67. chr10: 21,068,903-21,186,531 | 68. chr16: 69,743,304-69,760,533 |
| 69. chr2: 174,937,175-175,113,365 | 70. chr1: 207,101,867-207,119,811 |
| 71. chr1: 45,976,707-45,988,562 | 72. chr3: 93,591,881-93,692,934 |
| 73. chr9: 80,912,059-80,945,009 | 74. chr7: 105,096,960-105,162,685 |
| 75. chr19: 16,222,490-16,244,445 | 76. chr12: 112,842,994-112,847,443 |
| 77. chrX: 71,492,453-71,497,141 | 78. chr2: 3,622,853-3,628,509 |
| 79. chr1: 153,600,873-153,604,513 | 80. chr1: 153,507,076-153,508,717 |
| 81. chr4: 6,695,566-6,698,897 | 82. chr12: 56,623,820-56,631,629 |
| 83. chr16: 87,863,629-87,903,100 | 84. chr10: 105,727,470-105,787,342 |
| 85. chr21: 33,031,935-33,041,243 | 86. chr15: 45,315,302-45,367,287 |
| 87. chr1: 59,041,095-59,043,166 | 88. chr6: 160,199,530-160,210,735 |
| 89. chr3: 195,776,155-195,809,032 | 90. chr5: 135,364,584-135,399,507 |
| 91. chr6: 169,615,875-169,654,137 | 92. chr12: 27,124,506-27,167,339 |
| 93. chr18: 12,308,257-12,326,568 | 94. chr1: 7,831,329-7,841,492 |
| 95. chr17: 41,166,622-41,174,459 | |

in a membranous microvesicle sample from said individual wherein an increase in the level of expression of said genes relative to control levels is indicative of the onset or predisposition to the onset of a neoplasm.

In another aspect, said neoplasm is an adenoma or adenocarcinoma and even more preferably a colorectal adenoma or adenocarcinoma.

In yet another aspect, said method is directed to screening for the protein expression product or fragment thereof of said gene.

In still another aspect there is provided to a method of screening for the onset or predisposition to the onset of a large intestine neoplasm in an individual, said method comprising measuring the level of RNA transcripts transcribed from a gene selection from:

(i) any one or more genes selected from:

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 |
| 9. S100A11 | 10. ACAA2 | 11. ANPEP | 12. ANXA3 |
| 13. APP | 14. APPL2 | 15. AZGP1 | 16. BGN |
| 17. c20orf199 | 18. CALR | 19. CAP1 | 20. COL12A1 |
| 21. CSE1L | 22. CTSC | 23. CXCL3 | 24. DMBT1 |
| 25. ENO1 | 26. EPS8L3 | 27. FAT | 28. FTH1 |
| 29. GALNT6 | 30. GMDS | 31. GNB2L1 | 32. GPRC5A |
| 33. HEPH | 34. HLADRB1 | 35. HPGD | 36. HSP9OAA1 |
| 37. IFITM1 | 38. IFITM2 | 39. KRT8 | 40. LCN2 |
| 41. LDHB | 42. LIMA1 | 43. LOC440264 | 44. LRPPRC |
| 45. LRSAM1 | 46. MLLT3 | 47. MMP7 | 48. MUC13 |
| 49. MYO5B | 50. NDRG1 | 51. NEBL | 52. NQO1 |
| 53. OLA1 | 54. PIGR | 55. PRDX1 | 56. PROS1 |
| 57. PSAT1 | 58. PUS7 | 59. RAB8A | 60. RPL6 |
| 61. RPS4X | 62. RPS7 | 63. S100A1 | 64. S100A6 |
| 65. S100P | 66. SLC39A5 | 67. SLC7A5 | 68. SLK |
| 69. SOD1 | 70. SORD | 71. TACSTD2 | 72. TCP1 |
| 73. TFRC | 74. TGFBI | 75. THBS2 | 76. TM7SF3 |
| 77. TUBB6 | 78. VAMP3 | 79. VAT1; | |

or
(ii) any one or more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | 4. chr16: 89,687,000-89,704,839 |
| 5. chr12: 117,476,728-117,537, | 6. chr5: 127,419,483-127,525,380 |
| 251 | |
| 7. chr2: 173,292,314-173,371,181 | 8. chr1: 120,336,641-120,354,203 |
| 9. chr1: 152,004,982-152,009,511 | 10. chr18: 47,309,874-47,340,251 |
| 11. chr15: 90,328,126-90,358,072 | 12. chr4: 79,472,742-79,531,605 |
| 13. chr21: 27,252,861-27,543,138 | 14. chr12: 105,567,075-105,630,008 |
| 15. chr7: 99,564,350-99,573,735 | 16. chrX: 152,760,347-152,775,004 |
| 17. chr20: 47894715-47905797 | 18. chr20: 47894715-47905797 |
| 19. chr20: 47894715-47905797 | 20. chr20: 47894715-47905797 |
| 21. chr20: 47894808-47905797 | 22. chr20: 47895179-47905797 |
| 23. chr20: 47895179-47905797 | 24. chr19: 13,049,414-13,055,304 |
| 25. chr1: 40,506,255-40,538,321 | 26. chr6: 75,794,042-75,915,623 |
| 27. chr20: 47,662,838-47,713,486 | 28. chr11: 88,026,760-88,070,941 |
| 29. chr4: 74,902,312-74,904,490 | 30. chr10: 124,320,181-124,403,252 |
| 31. chr1: 8,921,059-8,939,151 | 32. chr1: 110,292,702-110,306,644 |
| 33. chr4: 126315091-126414087 | 34. chr5: 150935821-150948505 |
| 35. chr4: 187627717-187647850 | 36. chr4: 187508937-187516980 |
| 37. chr5: 150883653-150948505 | 38. chr5: 150883653-150911531 |
| 39. chr4: 187508937-187644987 | 40. chr4: 126237567-126414087 |
| 41. chr4: 126369616-126412943 | 42. chr11: 92085262-92629635 |
| 43. chr11: 92573728-92629635 | 44. chr11: 61,731,757-61,735,132 |
| 45. chr12: 51,745,833-51,785,200 | 46. chr6: 1,624,035-2,245,868 |
| 47. chr5: 180,663,928-180,670,906 | 48. chr12: 13,043,956-13,066,600 |
| 49. chrX: 65,382,433-65,487,230 | 50. chr6: 3,774,425-3,787,546 |
| 51. chr4: 175,411,328-175,443,792 | 52. chr14: 102547075-102606086 |
| 53. chr11: 313,991-315,272 | 54. chr11: 308,107-309,410 |
| 55. chr12: 53,290,971-53,298,868 | 56. chr9: 130,911,732-130,915,734 |
| 57. chr12: 21,788,275-21,810,789 | 58. chr12: 50,569,563-50,677,353 |
| 59. chr15: 30,262,050-30,265,947 | 60. chr2: 44,113,363-44,223,144 |
| 61. chr9: 130,214,534-130,265,780 | 62. chr9: 20,344,968-20,622,514 |
| 63. chr11: 102,391,239-102,401, | 64. chr3: 124,624,289-124,653,595 |
| 478 | |
| 65. chr18: 47,349,156-47,721,451 | 66. chr8: 134,249,414-134,309,547 |
| 67. chr10: 21,068,903-21,186,531 | 68. chr16: 69,743,304-69,760,533 |
| 69. chr2: 174,937,175-175,113,365 | 70. chr1: 207,101,867-207,119,811 |
| 71. chr1: 45,976,707-45,988,562 | 72. chr3: 93,591,881-93,692,934 |
| 73. chr9: 80,912,059-80,945,009 | 74. chr7: 105,096,960-105,162,685 |
| 75. chr19: 16,222,490-16,244,445 | 76. chr12: 112,842,994-112,847,443 |
| 77. chrX: 71,492,453-71,497,141 | 78. chr2: 3,622,853-3,628,509 |
| 79. chr1: 153,600,873-153,604,513 | 80. chr1: 153,507,076-153,508,717 |
| 81. chr4: 6,695,566-6,698,897 | 82. chr12: 56,623,820-56,631,629 |
| 83. chr16: 87,863,629-87,903,100 | 84. chr10: 105,727,470-105,787,342 |
| 85. chr21: 33,031,935-33,041,243 | 86. chr15: 45,315,302-45,367,287 |
| 87. chr1: 59,041,095-59,043,166 | 88. chr6: 160,199,530-160,210,735 |
| 89. chr3: 195,776,155-195,809,032 | 90. chr5: 135,364,584-135,399,507 |
| 91. chr6: 169,615,875-169,654,137 | 92. chr12: 27,124,506-27,167,339 |
| 93. chr18: 12,308,257-12,326,568 | 94. chr1: 7,831,329-7,841,492 |
| 95. chr17: 41,166,622-41,174,459 | |

in a membranous microvesicle sample from said individual wherein an increase in the level of expression of said RNA transcript relative to control levels is indicative of the onset or predisposition to the onset of a neoplasm.

In yet still another aspect, said RNA transcript is mRNA.

In still yet another aspect, said membranous microvesicles are exosomes.

In a further aspect, said gene is one or more of:

(i)

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 |
| 9. S100A11; | | | |

or
(ii) one or more of the regions defined by Hg19 coordinates:

1.chr15:81,071,712-81,243,999

2. chr16:54,952,778-54,963,079

3. chr13:53,602,876-53,626,196

4. chr16:89,687,000-89,704,839

5. chr12:117,476,728-117,537,251

6. chr5:127,419,483-127,525,380

7. chr2:173,292,314-173,371,181

8. chr1:120,336,641-120,354,203

9. chr 1:152,004,982-152,009,511

In still a further aspect, said region is one or more of

(i)

| | | |
|---|---|---|
| 1. KIAA1199 | 2. CRNDE | 3. OLFM4; |

or
(ii) one or more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | |

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

### BRIEF DESCRIPTION OF THE DRAWINGS

FIG. 1 is a graphical representation of GAPDH levels in human plasma (n=398).

FIG. 2 is a graphical representation depicting that KIAA1199 was detected more frequently in plasma from colorectal neoplastic patients (45 patient panel shown).

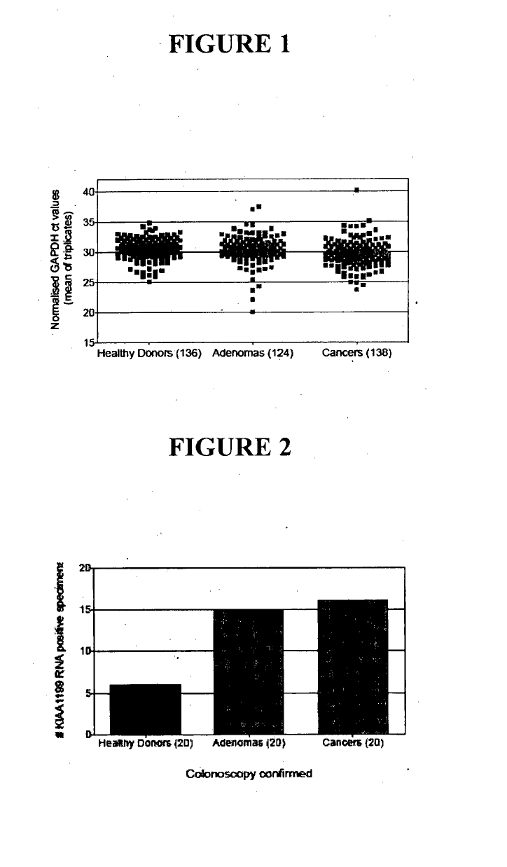

### DETAILED DESCRIPTION OF THE INVENTION

The present invention is predicated, in part, on the determination that within the panel of genes which are known to undergo increased expression in colorectal neoplasia patients there are, in fact, a small subgroup of genes which are detectable at increased levels in the membranous microvesicles, such as exosomes, of these patients. This determination is surprising. Although most genes which are expressed at increased levels in the tissue are expected to be detectable in plasma, particularly at the protein level, the fact is that the sensitivity of detection is not always adequate. Due to the inherent nature of exosomes, and the fact that they can now be reliably enriched for, there is provided a significantly more sensitive and accurate means to test for changes in the expression levels of these genes, at the RNA level, than just screening whole blood or plasma for protein or RNA. When considered together with the fact that exosomes can be conveniently harvested from blood samples, there is provided a particularly sensitive means of screening for colorectal neoplasia based on a minimally invasive protocol. This is highly relevant in the context of enabling early diagnosis of colorectal neoplasia.

Accordingly, one aspect of the present invention is directed to a method of screening for the onset or predisposition to the onset of a large intestine neoplasm in an individual, said method comprising measuring the level of expression of:

(i) any one or more genes selected from:

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 |
| 9. S100A11 | 10. ACAA2 | 11. ANPEP | 12. ANXA3 |
| 13. APP | 14. APPL2 | 15. AZGP1 | 16. BGN |
| 17. c20orf199 | 18. CALR | 19. CAP1 | 20. COL12A1 |
| 21. CSE1L | 22. CTSC | 23. CXCL3 | 24. DMBT1 |
| 25. ENO1 | 26. EPS8L3 | 27. FAT | 28. FTH1 |
| 29. GALNT6 | 30. GMDS | 31. GNB2L1 | 32. GPRC5A |
| 33. HEPH | 34. HLADRB1 | 35. HPGD | 36. HSP9OAA1 |
| 37. IFITM1 | 38. IFITM2 | 39. KRT8 | 40. LCN2 |
| 41. LDHB | 42. LIMA1 | 43. LOC440264 | 44. LRPPRC |
| 45. LRSAM1 | 46. MLLT3 | 47. MMP7 | 48. MUC13 |
| 49. MYO5B | 50. NDRG1 | 51. NEBL | 52. NQO1 |
| 53. OLA1 | 54. PIGR | 55. PRDX1 | 56. PROS1 |
| 57. PSAT1 | 58. PUS7 | 59. RAB8A | 60. RPL6 |
| 61. RPS4X | 62. RPS7 | 63. S100A1 | 64. S100A6 |
| 65. S100P | 66. SLC39A5 | 67. SLC7A5 | 68. SLK |
| 69. SOD1 | 70. SORD | 71. TACSTD2 | 72. TCP1 |
| 73. TFRC | 74. TGFBI | 75. THBS2 | 76. TM7SF3 |
| 77. TUBB6 | 78. VAMP3 | 79. VAT1; | |

or
(ii) any one or more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | 4. chr16: 89,687,000-89,704,839 |
| 5. chr12: 117,476,728-117,537, | 6. chr5: 127,419,483-127,525,380 |
| 251 | |
| 7. chr2: 173,292,314-173,371,181 | 8. chr1: 120,336,641-120,354,203 |
| 9. chr1: 152,004,982-152,009,511 | 10. chr18: 47,309,874-47,340,251 |
| 11. chr15: 90,328,126-90,358,072 | 12. chr4: 79,472,742-79,531,605 |
| 13. chr21: 27,252,861-27,543,138 | 14. chr12: 105,567,075-105,630,008 |
| 15. chr7: 99,564,350-99,573,735 | 16. chrX: 152,760,347-152,775,004 |
| 17. chr20: 47894715-47905797 | 18. chr20: 47894715-47905797 |
| 19. chr20: 47894715-47905797 | 20. chr20: 47894715-47905797 |
| 21. chr20: 47894808-47905797 | 22. chr20: 47895179-47905797 |
| 23. chr20: 47895179-47905797 | 24. chr19: 13,049,414-13,055,304 |
| 25. chr1: 40,506,255-40,538,321 | 26. chr6: 75,794,042-75,915,623 |
| 27. chr20: 47,662,838-47,713,486 | 28. chr11: 88,026,760-88,070,941 |
| 29. chr4: 74,902,312-74,904,490 | 30. chr10: 124,320,181-124,403,252 |
| 31. chr1: 8,921,059-8,939,151 | 32. chr1: 110,292,702-110,306,644 |
| 33. chr4: 126315091-126414087 | 34. chr5: 150935821-150948505 |
| 35. chr4: 187627717-187647850 | 36. chr4: 187508937-187516980 |
| 37. chr5: 150883653-150948505 | 38. chr5: 150883653-150911531 |
| 39. chr4: 187508937-187644987 | 40. chr4: 126237567-126414087 |
| 41. chr4: 126369616-126412943 | 42. chr11: 92085262-92629635 |
| 43. chr11: 92573728-92629635 | 44. chr11: 61,731,757-61,735,132 |
| 45. chr12: 51,745,833-51,785,200 | 46. chr6: 1,624,035-2,245,868 |
| 47. chr5: 180,663,928-180,670,906 | 48. chr12: 13,043,956-13,066,600 |
| 49. chrX: 65,382,433-65,487,230 | 50. chr6: 3,774,425-3,787,546 |
| 51. chr4: 175,411,328-175,443,792 | 52. chr14: 102547075-102606086 |
| 53. chr11: 313,991-315,272 | 54. chr11: 308,107-309,410 |
| 55. chr12: 53,290,971-53,298,868 | 56. chr9: 130,911,732-130,915,734 |
| 57. chr12: 21,788,275-21,810,789 | 58. chr12: 50,569,563-50,677,353 |
| 59. chr15: 30,262,050-30,265,947 | 60. chr2: 44,113,363-44,223,144 |
| 61. chr9: 130,214,534-130,265,780 | 62. chr9: 20,344,968-20,622,514 |
| 63. chr11: 102,391,239-102,401, | 64. chr3: 124,624,289-124,653,595 |
| 478 | |
| 65. chr18: 47,349,156-47,721,451 | 66. chr8: 134,249,414-134,309,547 |
| 67. chr10: 21,068,903-21,186,531 | 68. chr16: 69,743,304-69,760,533 |
| 69. chr2: 174,937,175-175,113,365 | 70. chr1: 207,101,867-207,119,811 |
| 71. chr1: 45,976,707-45,988,562 | 72. chr3: 93,591,881-93,692,934 |
| 73. chr9: 80,912,059-80,945,009 | 74. chr7: 105,096,960-10,162,685 |
| 75. chr19: 16,222,490-16,244,445 | 76. chr12: 112,842,994-112,847,443 |
| 77. chrX: 71,492,453-71,497,141 | 78. chr2: 3,622,853-3,628,509 |
| 79. chr1: 153,600,873-153,604,513 | 80. chr1: 153,507,076-153,508,717 |
| 81. chr4: 6,695,566-6,698,897 | 82. chr12: 56,623,820-56,631,629 |
| 83. chr16: 87,863,629-87,903,100 | 84. chr10: 105,727,470-105,787,342 |
| 85. chr21: 33,031,935-33,041,243 | 86. chr15: 45,315,302-45,367,287 |
| 87. chr1: 59,041,095-59,043,166 | 88. chr6: 160,199,530-160,210,735 |
| 89. chr3: 195,776,155-195,809,032 | 90. chr5: 135,364,584-135,399,507 |
| 91. chr6: 169,615,875-169,654,137 | 92. chr12: 27,124,506-27,167,339 |
| 93. chr18: 12,308,257-12,326,568 | 94. chr1: 7,831,329-7,841,492 |
| 95. chr17: 41,166,622-41,174,459 | |

in a membranous microvesicle sample from said individual wherein an increase in the level of expression of said genes relative to control levels is indicative of the onset or predisposition to the onset of a neoplasm.

It should be understood that the genes in issue are described herein both by reference to their name and their chromosomal coordinates. The chromosomal coordinates are consistent with the human genome database version Hg19 which was released in February 2009 (herein referred to as “Hg19 coordinates”).

Reference to “large intestine” should be understood as a reference to a cell derived from one of the six anatomical regions of the large intestine, which regions commence after the terminal region of the ileum, these being:

(i) the cecum;

(ii) the ascending colon;

(iii) the transverse colon;

(iv) the descending colon;

(v) the sigmoid colon; and

(vi) the rectum.

Reference to “neoplasm” should be understood as a reference to a lesion, tumour or other encapsulated or unencapsulated mass or other form of growth which comprises neoplastic cells. A “neoplastic cell” should be understood as a reference to a cell exhibiting abnormal growth. The term “growth” should be understood in its broadest sense and includes reference to proliferation. In this regard, an example of abnormal cell growth is the uncontrolled proliferation of a cell. Another example is failed apoptosis in a cell, thus prolonging its usual life span. The neoplastic cell may be a benign cell or a malignant cell. In a preferred embodiment, the subject neoplasm is an adenoma or an adenocarcinoma. Without limiting the present invention to any one theory or mode of action, an adenoma is generally a benign tumour of epithelial origin which is either derived from epithelial tissue or exhibits clearly defined epithelial structures. These structures may take on a glandular appearance. It can comprise a malignant cell population within the adenoma, such as occurs with the progression of a benign adenoma to a malignant adenocarcinoma.

The present invention is designed to screen for a neoplastic cell or cellular population, which is located within the large intestine. Accordingly, reference to “cell or cellular population” should be understood as a reference to an individual cell or a group of cells. Said group of cells may be a diffuse population of cells, a cell suspension, an encapsulated population of cells or a population of cells which take the form of tissue.

In one embodiment, said neoplasm is an adenoma or adenocarcinoma and even more preferably a colorectal adenoma or adenocarcinoma.

Reference to the genes detailed above (which are herein collectively referred to as “gene markers”) and their transcribed and translated expression products should be understood as a reference to all forms of these genes and proteins and to fragments thereof. As would be appreciated by the person of skill in the art, genes are known to exhibit allelic or polymorphic variation between individuals. Accordingly, reference to these genes should be understood to extend to such variants which, in terms of the present diagnostic applications, achieve the same outcome despite the fact that minor genetic variations between the actual nucleic acid sequences may exist between individuals. Splice variants of the gene markers may also commonly exist, this being a reference to alternative transcriptional forms of these genes which exhibit variation in exon expression and arrangement, such as in terms of multiple exon combinations or alternate 5′ or 3′-ends. The present invention should therefore be understood to extend to all forms of RNA (e.g. mRNA, primary RNA transcript, miRNA, etc), cDNA and isoforms which arise from alternative splicing or any other mutation, polymorphic or allelic variation. It should also be understood to include reference to any subunit polypeptides such as precursor forms.

In terms of the method of the present invention, screening for the “level of expression” of these gene markers may be achieved in a variety of ways including screening for any of the forms of RNA transcribed from these genes or cDNA generated therefrom. Reference to “screening for the level of RNA transcripts” should be understood as a reference to either screening the RNA directly or screening cDNA transcribed therefrom. Changes to the levels of any of these products is indicative of changes to the expression of the subject gene. Still further, the nucleic acid molecule which is identified and measured may be a whole molecule or a fragment thereof. For example, one may identify only fragments of RNA from an exosome sample, depending on how it has been processed. Provided that said fragment comprises sufficient sequence to indicate its origin with a particular gene, fragmented gene molecules are useful in the context of the method of the present invention.

It should also be understood that the level of expression may be assessed by screening for the level of expression of the subject gene's protein expression product, including fragments thereof, within the membranous microvesicle. The protein sequences for the genes described herein are well known and routinely obtainable by the skilled person from publically accessible databases. Nevertheless, provided herein are the protein sequences for KIA1199 (SEQ ID NO: 1), OLFM4 (SEQ ID NO: 2), DPEP1 (SEQ ID Nos: 3 and 4), S100A11 (SEQ ID NO: 5), ITGA6 (SEQ ID NOs: 6 and 7), TESC (SEQ ID NOs: 8 and 9), REG4 (SEQ ID NOs: 10, 11 and 12) and SLC12A2 (SEQ ID NO: 13).

In one embodiment, said method is directed to screening for the protein expression product or fragment thereof of said gene.

Reference to “nucleic acid molecule” should be understood as a reference to both deoxyribonucleic acid molecules and ribonucleic acid molecules and fragments thereof. The present invention therefore extends to both directly screening for RNA levels in an exosome sample or screening for the complementary cDNA which has been reverse-transcribed from an RNA population of interest. It is well within the skill of the person of skill in the art to design methodology directed to screening for either DNA or RNA.

Reference to a “fragment” should be understood as a reference to a portion of the subject gene or nucleic acid molecule. As detailed hereinbefore, this is particularly relevant with respect to screening for modulated RNA levels in exosome samples which may have been enzymatically treated since the subject RNA may have been degraded or otherwise fragmented. One may therefore actually be detecting fragments of the subject RNA molecule, which fragments are identified by virtue of the use of a suitably specific probe.

In another embodiment there is provided to a method of screening for the onset or predisposition to the onset of a large intestine neoplasm in an individual; said method comprising measuring the level of RNA transcripts transcribed from a gene selection from:

(i) any one or more genes selected from:

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 |
| 9. S100A11 | 10. ACAA2 | 11. ANPEP | 12. ANXA3 |
| 13. APP | 14. APPL2 | 15. AZGP1 | 16. BGN |
| 17. c20orf199 | 18. CALR | 19. CAP1 | 20. COL12A1 |
| 21. CSE1L | 22. CTSC | 23. CXCL3 | 24. DMBT1 |
| 25. ENO1 | 26. EPS8L3 | 27. FAT | 28. FTH1 |
| 29. GALNT6 | 30. GMDS | 31. GNB2L1 | 32. GPRC5A |
| 33. HEPH | 34. HLADRB1 | 35. HPGD | 36. HSP9OAA1 |
| 37. IFITM1 | 38. IFITM2 | 39. KRT8 | 40. LCN2 |
| 41. LDHB | 42. LIMA1 | 43. LOC440264 | 44. LRPPRC |
| 45. LRSAM1 | 46. MLLT3 | 47. MMP7 | 48. MUC13 |
| 49. MYO5B | 50. NDRG1 | 51. NEBL | 52. NQO1 |
| 53. OLA1 | 54. PIGR | 55. PRDX1 | 56. PROS1 |
| 57. PSAT1 | 58. PUS7 | 59. RAB8A | 60. RPL6 |
| 61. RPS4X | 62. RPS7 | 63. S100A1 | 64. S100A6 |
| 65. S100P | 66. SLC39A5 | 67. SLC7A5 | 68. SLK |
| 69. SOD1 | 70. SORD | 71. TACSTD2 | 72. TCP1 |
| 73. TFRC | 74. TGFBI | 75. THBS2 | 76. TM7SF3 |
| 77. TUBB6 | 78. VAMP3 | 79. VAT1; | |

or
(ii) any one or more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | 4. chr16: 89,687,000-89,704,839 |
| 5. chr12: 117,476,728-117,537, | 6. chr5: 127,419,483-127,525,380 |
| 251 | |
| 7. chr2: 173,292,314-173,371,181 | 8. chr1: 120,336,641-120,354,203 |
| 9. chr1: 152,004,982-152,009,511 | 10. chr18: 47,309,874-47,340,251 |
| 11. chr15: 90,328,126-90,358,072 | 12. chr4: 79,472,742-79,531,605 |
| 13. chr21: 27,252,861-27,543,138 | 14. chr12: 105,567,075-105,630,008 |
| 15. chr7: 99,564,350-99,573,735 | 16. chrX: 152,760,347-152,775,004 |
| 17. chr20: 47894715-47905797 | 18. chr20: 47894715-47905797 |
| 19. chr20: 47894715-47905797 | 20. chr20: 47894715-47905797 |
| 21. chr20: 47894808-47905797 | 22. chr20: 47895179-47905797 |
| 23. chr20: 47895179-47905797 | 24. chr19: 13,049,414-13,055,304 |
| 25. chr1: 40,506,255-40,538,321 | 26. chr6: 75,794,042-75,915,623 |
| 27. chr20: 47,662,838-47,713,486 | 28. chr11: 88,026,760-88,070,941 |
| 29. chr4: 74,902,312-74,904,490 | 30. chr10: 124,320,181-124,403,252 |
| 31. chr1: 8,921,059-8,939,151 | 32. chr1: 110,292,702-110,306,644 |
| 33. chr4: 126315091-126414087 | 34. chr5: 150935821-150948505 |
| 35. chr4: 187627717-187647850 | 36. chr4: 187508937-187516980 |
| 37. chr5: 150883653-150948505 | 38. chr5: 150883653-150911531 |
| 39. chr4: 187508937-187644987 | 40. chr4: 126237567-126414087 |
| 41. chr4: 126369616-126412943 | 42. chr11: 92085262-92629635 |
| 43. chr11: 92573728-92629635 | 44. chr11: 61,731,757-61,735,132 |
| 45. chr12: 51,745,833-51,785,200 | 46. chr6: 1,624,035-2,245,868 |
| 47. chr5: 180,663,928-180,670,906 | 48. chr12: 13,043,956-13,066,600 |
| 49. chrX: 65,382,433-65,487,230 | 50. chr6: 3,774,425-3,787,546 |
| 51. chr4: 175,411,328-175,443,792 | 52. chr14: 102547075-102606086 |
| 53. chr11: 313,991-315,272 | 54. chr11: 308,107-309,410 |
| 55. chr12: 53,290,971-53,298,868 | 56. chr9: 130,911,732-130,915,734 |
| 57. chr12: 21,788,275-21,810,789 | 58. chr12: 50,569,563-50,677,353 |
| 59. chr15: 30,262,050-30,265,947 | 60. chr2: 44,113,363-44,223,144 |
| 61. chr9: 130,214,534-130,265,780 | 62. chr9: 20,344,968-20,622,514 |
| 63. chr11: 102,391,239-102,401, | 64. chr3: 124,624,289-124,653,595 |
| 478 | |
| 65. chr18: 47,349,156-47,721,451 | 66. chr8: 134,249,414-134,309,547 |
| 67. chr10: 21,068,903-21,186,531 | 68. chr16: 69,743,304-69,760,533 |
| 69. chr2: 174,937,175-175,113,365 | 70. chr1: 207,101,867-207,119,811 |
| 71. chr1: 45,976,707-45,988,562 | 72. chr3: 93,591,881-93,692,934 |
| 73. chr9: 80,912,059-80,945,009 | 74. chr7: 105,096,960-105,162,685 |
| 75. chr19: 16,222,490-16,244,445 | 76. chr12: 112,842,994-112,847,443 |
| 77. chrX: 71,492,453-71,497,141 | 78. chr2: 3,622,853-3,628,509 |
| 79. chr1: 153,600,873-153,604,513 | 80. chr1: 153,507,076-153,508,717 |
| 81. chr4: 6,695,566-6,698,897 | 82. chr12: 56,623,820-56,631,629 |
| 83. chr16: 87,863,629-87,903,100 | 84. chr10: 105,727,470-105,787,342 |
| 85. chr21: 33,031,935-33,041,243 | 86. chr15: 45,315,302-45,367,287 |
| 87. chr1: 59,041,095-59,043,166 | 88. chr6: 160,199,530-160,210,735 |
| 89. chr3: 195,776,155-195,809,032 | 90. chr5: 135,364,584-135,399,507 |
| 91. chr6: 169,615,875-169,654,137 | 92. chr12: 27,124,506-27,167,339 |
| 93. chr18: 12,308,257-12,326,568 | 94. chr1: 7,831,329-7,841,492 |
| 95. chr17: 41,166,622-41,174,459 | |

in a membranous microvesicle sample from said individual wherein an increase in the level of expression of said RNA transcript relative to control levels is indicative of the onset or predisposition to the onset of a neoplasm.

In one embodiment, said RNA transcript is mRNA.

Reference to “membranous microvesicle” should be understood as a reference to any particle which is comprised of a cellular plasma membrane component. Said membranous microvesicles may adopt a structure which takes the form of a lumen surrounded by plasma membrane. Examples of membranous microvesicles include, but are not limited to, microparticles, exosomes, apoptotic blebs, apoptotic bodies, cellular blebs and the like. In one embodiment, said membranous microvesicles are exosomes.

Accordingly, another aspect of the present invention is directed to a method of screening for the onset or predisposition to the onset of a large intestine neoplasm in an individual, said method comprising measuring the level of expression of:

(i) any one or more genes selected from:

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 |
| 9. S100A11 | 10. ACAA2 | 11. ANPEP | 12. ANXA3 |
| 13. APP | 14. APPL2 | 15. AZGP1 | 16. BGN |
| 17. c20orf199 | 18. CALR | 19. CAP1 | 20. COL12A1 |
| 21. CSE1L | 22. CTSC | 23. CXCL3 | 24. DMBT1 |
| 25. ENO1 | 26. EPS8L3 | 27. FAT | 28. FTH1 |
| 29. GALNT6 | 30. GMDS | 31. GNB2L1 | 32. GPRC5A |
| 33. HEPH | 34. HLADRB1 | 35. HPGD | 36. HSP9OAA1 |
| 37. IFITM1 | 38. IFITM2 | 39. KRT8 | 40. LCN2 |
| 41. LDHB | 42. LIMA1 | 43. LOC440264 | 44. LRPPRC |
| 45. LRSAM1 | 46. MLLT3 | 47. MMP7 | 48. MUC13 |
| 49. MYO5B | 50. NDRG1 | 51. NEBL | 52. NQO1 |
| 53. OLA1 | 54. PIGR | 55. PRDX1 | 56. PROS1 |
| 57. PSAT1 | 58. PUS7 | 59. RAB8A | 60. RPL6 |
| 61. RPS4X | 62. RPS7 | 63. S100A1 | 64. S100A6 |
| 65. S100P | 66. SLC39A5 | 67. SLC7A5 | 68. SLK |
| 69. SOD1 | 70. SORD | 71. TACSTD2 | 72. TCP1 |
| 73. TFRC | 74. TGFBI | 75. THBS2 | 76. TM7SF3 |
| 77. TUBB6 | 78. VAMP3 | 79. VAT1; | |

or
(ii) any one or more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | 4. chr16: 89,687,000-89,704,839 |
| 5. chr12: 117,476,728-117,537, | 6. chr5: 127,419,483-127,525,380 |
| 251 | |
| 7. chr2: 173,292,314-173,371,181 | 8. chr1: 120,336,641-120,354,203 |
| 9. chr1: 152,004,982-152,009,511 | 10. chr18: 47,309,874-47,340,251 |
| 11. chr15: 90,328,126-90,358,072 | 12. chr4: 79,472,742-79,531,605 |
| 13. chr21: 27,252,861-27,543,138 | 14. chr12: 105,567,075-105,630,008 |
| 15. chr7: 99,564,350-99,573,735 | 16. chrX: 152,760,347-152,775,004 |
| 17. chr20: 47894715-47905797 | 18. chr20: 47894715-47905797 |
| 19. chr20: 47894715-47905797 | 20. chr20: 47894715-47905797 |
| 21. chr20: 47894808-47905797 | 22. chr20: 47895179-47905797 |
| 23. chr20: 47895179-47905797 | 24. chr19: 13,049,414-13,055,304 |
| 25. chr1: 40,506,255-40,538,321 | 26. chr6: 75,794,042-75,915,623 |
| 27. chr20: 47,662,838-47,713,486 | 28. chr11: 88,026,760-88,070,941 |
| 29. chr4: 74,902,312-74,904,490 | 30. chr10: 124,320,181-124,403,252 |
| 31. chr1: 8,921,059-8,939,151 | 32. chr1: 110,292,702-110,306,644 |
| 33. chr4: 126315091-126414087 | 34. chr5: 150935821-150948505 |
| 35. chr4: 187627717-187647850 | 36. chr4: 187508937-187516980 |
| 37. chr5: 150883653-150948505 | 38. chr5: 150883653-150911531 |
| 39. chr4: 187508937-187644987 | 40. chr4: 126237567-126414087 |
| 41. chr4: 126369616-126412943 | 42. chr11: 92085262-92629635 |
| 43. chr11: 92573728-92629635 | 44. chr11: 61,731,757-61,735,132 |
| 45. chr12: 51,745,833-51,785,200 | 46. chr6: 1,624,035-2,245,868 |
| 47. chr5: 180,663,928-180,670,906 | 48. chr12: 13,043,956-13,066,600 |
| 49. chrX: 65,382,433-65,487,230 | 50. chr6: 3,774,425-3,787,546 |
| 51. chr4: 175,411,328-175,443,792 | 52. chr14: 102547075-102606086 |
| 53. chr11: 313,991-315,272 | 54. chr11: 308,107-309,410 |
| 55. chr12: 53,290,971-53,298,868 | 56. chr9: 130,911,732-130,915,734 |
| 57. chr12: 21,788,275-21,810,789 | 58. chr12: 50,569,563-50,677,353 |
| 59. chr15: 30,262,050-30,265,947 | 60. chr2: 44,113,363-44,223,144 |
| 61. chr9: 130,214,534-130,265,780 | 62. chr9: 20,344,968-20,622,514 |
| 63. chr11: 102,391,239-102,401, | 64. chr3: 124,624,289-124,653,595 |
| 478 | |
| 65. chr18: 47,349,156-47,721,451 | 66. chr8: 134,249,414-134,309,547 |
| 67. chr10: 21,068,903-21,186,531 | 68. chr16: 69,743,304-69,760,533 |
| 69. chr2: 174,937,175-175,113,365 | 70. chr1: 207,101,867-207,119,811 |
| 71. chr1: 45,976,707-45,988,562 | 72. chr3: 93,591,881-93,692,934 |
| 73. chr9: 80,912,059-80,945,009 | 74. chr7: 105,096,960-105,162,685 |
| 75. chr19: 16,222,490-16,244,445 | 76. chr12: 112,842,994-112,847,443 |
| 77. chrX: 71,492,453-71,497,141 | 78. chr2: 3,622,853-3,628,509 |
| 79. chr1: 153,600,873-153,604,513 | 80. chr1: 153,507,076-153,508,717 |
| 81. chr4: 6,695,566-6,698,897 | 82. chr12: 56,623,820-56,631,629 |
| 83. chr16: 87,863,629-87,903,100 | 84. chr10: 105,727,470-105,787,342 |
| 85. chr21: 33,031,935-33,041,243 | 86. chr15: 45,315,302-45,367,287 |
| 87. chr1: 59,041,095-59,043,166 | 88. chr6: 160,199,530-160,210,735 |
| 89. chr3: 195,776,155-195,809,032 | 90. chr5: 135,364,584-135,399,507 |
| 91. chr6: 169,615,875-169,654,137 | 92. chr12: 27,124,506-27,167,339 |
| 93. chr18: 12,308,257-12,326,568 | 94. chr1: 7,831,329-7,841,492 |
| 95. chr17: 41,166,622-41,174,459 | |

in an exosome sample from said individual wherein an increase in the level of expression of said genes relative to control levels is indicative of the onset or predisposition to the onset of a neoplasm.

In one embodiment said large intestine neoplasia is a colorectal adenoma or adenocarcinoma.

In another embodiment, said level of gene expression is the level of RNA transcripts, such as mRNA.

In still another embodiment said method is directed to screening for the protein expression product or fragment thereof of said gene.

Reference to “exosome” should be understood as a reference to the vesicles which are secreted by a wide variety of cell types. Without limiting the present invention to any one theory or mode of action, late endosomes or multivesicular bodies contain intralumenal vesicles which are formed by the inward budding and scission of vesicles from the limited endosomal membrane into these enclosed nanovesicles. These intralumenal vesicles are then released from the multivesicular body lumen into the extracellular environment during exocytosis upon fusion with the plasma membrane. An exosome is created intracellularly when a segment of membrane invaginates and is endocytosed. The internalised segments which are broken into smaller vesicles and ultimately expelled from the cell contain proteins and RNA molecules such as mRNA and miRNA. Since plasma-derived exosomes largely lack ribosomal RNA, they are a useful source of RNA, in particular since it has now been determined that some of the increased gene expression which is observed in colorectal neoplasias is reflected in circulating exosome populations.

The exosomes of the present invention are enriched from a biological sample. By “biological sample” is meant any biological material derived from an individual. Such samples include, but are not limited to, blood, serum, plasma, urine, lymph, cerebrospinal fluid, ascites, saliva, mucus, stool, biopsy specimens, breast milk, gastric juice, pleural fluid, semen, sweat, tears, hair, vaginal secretion and fluid which has been introduced into the body of an individual and subsequently removed such as, for example, the saline solution extracted from the lung following lung lavage or the solution retrieved from an enema wash. The biological sample which is tested according to the method of the present invention may be tested directly or may require some form of pre-treatment prior to testing. For example, the sample may require the addition of a reagent, such as a buffer, to mobilise the sample. It should be further understood that the sample which is the subject of testing may be freshly isolated or it may have been isolated at an earlier point in time and subsequently stored or otherwise treated prior to testing. For example, the sample may have been collected at an earlier point in time and frozen or otherwise preserved in order to facilitate its transportation to the site of testing. In yet another example, the sample may be treated to neutralise any possible pathogenic infection, thereby reducing the risk of transmission of the infection to the technician.

In one embodiment, said biological sample is a blood, serum, plasma, urine, stool, saliva, tears or ascites fluid sample.

To the extent that the subject biological sample is harvested from an individual, the term “individual” should be understood to include a human, primate, livestock animal (e.g. sheep, pig, cow, horse, donkey), laboratory test animal (e.g. mouse, rat, rabbit, guinea pig), companion animal (e.g. dog, cat), captive wild animal (e.g. fox, kangaroo, deer), ayes (e.g. chicken, geese, duck, emu, ostrich), reptile or fish. Preferably, the subject individual is a human.

In another embodiment, said gene is one or more of:

(i)

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 |
| 9. S100A11; | | | |

or
(ii) one or more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | 4. chr16: 89,687,000-89,704,839 |
| 5. chr12: 117,476,728-117,537,251 | 6. chr5: 127,419,483-127,525,380 |
| 7. chr2: 173,292,314-173,371,181 | 8. chr1: 120,336,641-120,354,203 |
| 9. chr1: 152,004,982-152,009,511 | |

In another embodiment, said region is one or more of

(i)

| | | |
|---|---|---|
| 1. KIAA1199 | 2. CRNDE | 3. OLFM4; |

or
(ii) one or more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | |

The method of the present invention is predicated on the comparison of the level of expression of said gene markers in an exosome sample with the control levels of these genes. The “control level” is the “normal level”, which is the level of gene expressed by a corresponding exosome population from a normal individual.

The normal (or “non-neoplastic”) level may be determined using any suitable method, such as the analysis of test results relative to a standard result which reflects individual or collective results obtained from individuals other than the patient in issue. This form of analysis is in fact a preferred method of analysis since it enables the design of kits which require the collection and analysis of a single exosome sample, being a test sample of interest, relative to a predetermined standard. The standard results which provide the normal level may be calculated by any suitable means which would be well known to the person of skill in the art. For example, a population of normal plasma derived exosomes can be assessed in terms of the level of the gene marker in issue, thereby providing a standard value or range of values against which all future test samples are analysed. It should also be understood that the normal level may be determined from the subjects of a specific cohort and for use with respect to test samples derived from that cohort. Accordingly, there may be determined a number of standard values or ranges which correspond to cohorts which differ in respect of characteristics such as age, gender, ethnicity or health status. Said “normal level” may be a discrete level or a range of levels. An increase in the expression level of the subject gene marker relative to normal levels is indicative of the tissue being neoplastic.

Preferably, said control level is a non-neoplastic level.

According to these aspects of the present invention, said large intestine tissue is preferably colorectal tissue.

Still more preferably, said neoplasm is a colorectal adenoma or adenocarcinoma.

To the extent that the gene marker transcription product is present in an exosome sample, the biological sample may be directly tested or else all or some of the nucleic acid material present in the exosome sample may be isolated prior to testing. To this end, and as hereinbefore described, it would be appreciated that when screening for changes to the level of expression of said gene markers one may screen for the RNA transcripts themselves or cDNA which has been transcribed therefrom. It is within the scope of the present invention for the exosome population or molecules derived therefrom to be pretreated prior to testing, for example, inactivation of live virus or being run on a gel. It should also be understood that the exosome sample may be freshly harvested or it may have been stored (for example by freezing) prior to testing or otherwise treated prior to testing.

The choice of what type of sample is most suitable for testing in accordance with the method disclosed herein will be dependent on the nature of the situation.

Reference to the “onset” of a neoplasm, such as adenoma or adenocarcinoma, should be understood as a reference to one or more cells of that individual exhibiting dysplasia. In this regard, the adenoma or adenocarcinoma may be well developed in that a mass of dysplastic cells has developed. Alternatively, the adenoma or adenocarcinoma may be at a very early stage in that only relatively few abnormal cell divisions have occurred at the time of diagnosis. The present invention also extends to the assessment of an individual's predisposition to the development of a neoplasm, such as an adenoma or adenocarcinoma. Without limiting the present invention in any way, changed levels of the gene marker may be indicative of that individual's predisposition to developing a neoplasia, such as the future development of an adenoma or adenocarcinoma or another adenoma or adenocarcinoma.

Although the preferred method is to diagnose neoplasia development or predisposition thereto, the detection of converse changes in the levels of said marker may be desired under certain circumstances, for example, to monitor the effectiveness of therapeutic or prophylactic treatment directed to modulating a neoplastic condition, such as adenoma or adenocarcinoma development. For example, where elevated levels of the gene markers indicates that an individual has developed a condition characterised by adenoma or adenocarcinoma development, screening for a decrease in the levels of this marker subsequently to the onset of a therapeutic regime may be utilised to indicate reversal or other form of improvement of the subject individual's condition.

The method of the present invention is therefore useful as a one time test or as an on-going monitor of those individuals thought to be at risk of neoplasia development or as a monitor of the effectiveness of therapeutic or prophylactic treatment regimes directed to inhibiting or otherwise slowing neoplasia development. In these situations, mapping the modulation of gene marker expression levels in exosomes is a valuable indicator of the status of an individual or the effectiveness of a therapeutic or prophylactic regime which is currently in use. Accordingly, the method of the present invention should be understood to extend to monitoring for changes in gene marker expression levels in an individual relative to their normal level (as hereinbefore defined), or relative to one or more earlier marker expression levels determined from a biological sample of said individual.

The exosome sample may be derived from any suitable biological sample and may be either isolated from that sample or enriched for. Methods for performing isolation or enrichment are known and it is within the skill of the person in the art to select and apply a method appropriate to the particular circumstances. For example, exosomes may be enriched for by subjecting the biological sample of which they are part to mechanical rupture such that cellular material is ruptured and enzymatically cleared but not the exosomes. Due to differences in the physical characteristics of cells relative to exosomes, mechanical cellular rupture methods can be designed such that they exhibit sufficient force to disrupt a cell but not an exosome. This is due to the significant difference in physical characteristics such as the relatively larger mass of cells relative to exosomes. Since methods for examining a biological sample to identify the presence of intact cells or exosomes are extremely simple and routine, means for optimising any of the widely known standard techniques for mechanical cell rupture, to ensure that exosomes are not also ruptured, is a matter of routine procedure. Similarly, optimising any newly developed techniques would also be straightforward.

Methods of achieving mechanical cellular rupture are well known in the art and include, but are not limited to:
(i) centrifugation
(ii) sonication (with or without the inclusion of surfactants)
(iii) bead milling with or without the addition of surfactants using, for example, small glass, ceramic, zirconium or steel beads
(iv) homogenization
(v) nitrogen burst method
(vi) small probe ultrasound
(vii) hypotonic shock
(viii) High-shear mechanical methods;
(ix) rotor-stator disruptors,
(x) valve-type processors,
(xi) fixed geometry processors,
(xii) constant pressure processors,
(xiii) osmosis based electroporation, and
(xiv) electropermeabilization.

To the extent that the subject biological sample is a blood or plasma sample, or any other biological sample which either naturally or otherwise contains enzymes which degrade a molecule of diagnostic interest e.g. this enrichment method will conveniently achieve enrichment of the exosome population relative to not only the cellular population but, also, relative to the non-exosome proteinaceous and non-proteinaceous material in that sample.

Since the diagnostic method of the present invention requires amplification or sequencing of exosome nucleic acid material in order to detect the presence of the gene marker of interest, use of the enrichment method hereinbefore described will mean that there will not be a need to further purify the subject biological sample since techniques directed to analysing nucleic acid material are selective in this regard and provided that non-exosome nucleic acid material has been degraded, accurate results will be obtained. This enrichment method achieves both removal of unwanted cellular material without damaging exosome structure and, further, degradation of contaminating nucleic acid molecules due to nucleases which are naturally present in plasma, prior to analysis of the exosome-derived nucleic acids.

Although in its standard application this enrichment method may use the application of centrifugal force to separate components within a sample based on density, it is primarily designed to selectively rupture cells, rather than just pushing them into a pellet and then decanting/harvesting the supernatant which contains the exosomes. If appropriate centrifugal forces which rupture cells are not used, then even if the supernatant is separated from the pellet, it may still contain contaminating cells which retain their nucleic acid content. That being the case, since the purpose of harvesting the exosome population is to analyse its RNA, this will necessarily lead to aberrant results since all the steps designed to preserve and harvest the exosome RNA would equally preserve and harvest the RNA of the intact cells remaining in solution. However, by applying forces which selectively rupture cells, all cells are lysed and therefore the exosome population is heavily enriched. It is therefore not necessary to separate the supernatant from any pellet which may have formed since any such pellet will not be comprised of whole cells. Such an additional separation step would therefore be superfluous.

Even to the extent that it is sought to analyse the exosomal RNA, the fact that the exosomes remain in solution with degraded cellular material is of little consequence since the newly exposed cellular nucleic acid will be degraded by enzymes either naturally present in, or added to, the biological sample. Accordingly, no further enrichment or purification need be performed. It should be understood, however, that this does not exclude the performance of any additional steps. For example, one might want to perform one or more spins to pellet out and remove a proportion of the most dense particulate material present in the sample and to thereafter perform the diagnostic method on the supernatant harvested therefrom. However, the unique advantage of this particular enrichment technique is that this is not, in fact, necessary. Nevertheless, it is well within the skill of the person of skill in the art to determine both what type of sample to use and the nature of its mode of preparation prior to application of the present diagnostic method and, further, how to treat the enriched exosome population subsequently to its enrichment.

As detailed hereinbefore it should be understood that subsequently to mechanical cellular rupturing, there may still be left in solution some contaminants (i.e., non-exosome molecules). To the extent that these contaminants are nucleic acid molecules, such as DNA and RNA, they can be conveniently removed. Similarly, proteins can also be removed. This can be achieved via the use of enzymes such as nucleases and proteases. Provided that the exosomes themselves have not been lysed for the purpose of accessing their nucleic acid or protein content, this provides a convenient means of further purifying the sample which is obtained by the method of the present invention. To this end, it has been observed that in at least plasma samples, there are sufficient ribonucleases present to degrade free RNA, such as cytosolic derived RNA released due to breakage of contaminating cells subsequently to the mechanical rupturing step. Since this method ensures that the integrity of the exosomes, although not the cells, is maintained, to the extent that the RNA contained within the exosomes is of ultimate interest, this provides a convenient means to remove contaminating free RNA such that the results obtained from analysis of the exosomal derived RNA are accurate. It should be understood that if insufficient amounts of functional nucleases (DNAses or ribonucleases), or even proteinases, are naturally present in the sample, these molecules can be introduced into the sample at any suitable time point, such as prior to commencement of the mechanical cellular rupture process or part way through.

Other methods of purifying exosomes include well known prior art techniques such as density based separation techniques, filtration or membrane antigen-specific affinity isolation.

To the extent that it is sought to isolate and analyse the mRNA within the exosome, for example to assess changes to gene marker expression levels, it is necessary to lyse the exosome in order to expose its nucleic acid content and to thereafter analyse the mRNA subpopulation of nucleic acid molecules. To this end, the analysis of exosome RNA is often based on isolation of total RNA followed by PCR amplification of specific transcripts of interest. Methods for isolating and analysing total RNA are well known.

There are a wide variety of methods which can be and have been used to isolate total RNA from exosomes. The first step in isolating total RNA from such exosomes is to break open the exosome under denaturing conditions. The methods which are utilised mirror the methods used to isolate RNA from cells. Chirgwin et al. Biochemistry. 18(24):5294-9, 1979) devised a method for the efficient isolation of total RNA by homogenization in a 4 M solution of the protein denaturant guanidinium thiocyanate with 0.1 M 2-mercaptoethanol to break protein disulfide bonds. Chirgwin then isolated RNA by ethanol extraction or by ultracentrifugation through cesium chloride. Chomczynski and Sacchi (Analytical Biochemistry, 162(1):156-9, 1987) modified this method to devise a rapid single-step extraction procedure using a mixture of guanidinium thiocyanate and phenol-chloroform, a method especially useful for processing large numbers of samples or for isolation of RNA from small quantities of cells or tissue.

Many of the kits currently available are based on these two methods, with proprietary mixes of guanidinium thiocyanate and phenol-chloroform for optimum results. Alternative lysis methods may also be used such as detergent lysis and organic extraction replaced with adsorption to an affinity matrix.

Access to isolated nucleic acids requires both cell lysis and inactivation of cellular nucleases, a process that must be harsh enough to break open cells, but gentle enough to result in intact nucleic acids. This may be achieved mechanically, by homogenization, or chemically, by detergent lysis or chaotropic agents. In most procedures, lysis and inactivation are achieved by a single solution. For example, TRIzol reagent, manufactured by Molecular Research Center Inc. and used in Life Technologies' MessageMaker® mRNA Isolation System, is a mixture of acidic phenol and guanidine isothiocyanate. Tissue samples are lysed in TRIzol, and total RNA is obtained by chloroform extraction and isopropanol precipitation. Similarly, Chaosolv, used in ULTRASPEC® RNA isolation kits from BIOTECX Laboratories Inc., is a 14 M solution of guanidine salts and urea, which acts as a denaturing agent and is used in conjunction with phenol and other detergents.

For cells and tissue from which RNA is difficult to isolate by conventional methods, Bio101 offers the FastPrep System. This system is based on a benchtop instrument that uses a rapid reciprocating motion and a combination of matrices and chaotropic reagents to simultaneously homogenize tissue, lyse cells, and stabilize RNA in a matter of seconds. Rapid agitation of the lysing matrix leads to efficient lysis of a wide range of material. Each FastRNA® kit, designed to isolate RNA from specific cell and tissue types, contains a different lysing matrix: silica particles (for bacteria), ceramic particles (for yeast, fungi, and algae), and zirconium particles (for plant and animal tissue).

Silica- or glass-based matrices or filters are popular choices for selective adsorption of RNA. Total RNA binds to the matrices or filters in the presence of chaotropic salts, usually enabling the user to avoid using organic solvents for extraction from lysates.

Ambion's RNAqueous systems rely on binding of RNA to a glass-fiber filter. In the standard RNAqueous kit, designed for small-scale applications, the filter is housed in a filter cartridge in a microfuge tube. Solutions are driven through the filter by centrifugation or under vacuum. For larger applications, the filter is housed in a luer lock syringe filter in the RNAqueous-MIDI kit. Solutions can be pushed through the glass-fiber filter using a 10- or 20-ml syringe. To process several samples at once, the syringe filter units can be fitted onto a vacuum manifold.

Bio101's RNaid Plus kits include the proprietary silica gel-based RNAMATRIX®. Prior to binding RNA to the RNAMATRIX, this protocol does require an acid phenol extraction of the lysate. RNA binding is in a batch format and the spin modules are used to separate eluted RNA from the matrix.

Using a reverse binding strategy, Bioline Ltd.'s RNAce kits are used to isolate RNA from cell lysates by binding contaminating DNA to a mineral carrier. The resulting supernatant contains undegraded RNA that is free from contaminating DNA.

CLONTECH offers NucleoSpin® RNA II and NucleoTrap mRNA kits, both based on purification of RNA via a silica support. NucleoSpin columns contain a unique silica membrane that binds DNA and RNA in the presence of chaotropic salt. DNA is removed from the preparation by adding DNase I directly to the column. NucleoTrap is an activated spherical silica matrix in suspension that binds RNA.

S.N.A.P. is a silica-based resin available from Invitrogen Corp. In the S.N.A.P. Total RNA Isolation Kit, the resin comes in a membrane/column format, which allows for efficient multiple sample processing.

Life Technologies' GLASSMAX RNA Isolation Spin Cartridges contain a negatively charged silica matrix that binds RNA. Cells are lysed in guanidine isothiocyanate, and the sample suspended in an acid sodium solution. This is applied to the spin cartridges, from which bound RNA can then be eluted.

QIAGEN's RNeasy kits combine the advantages of guanidinium thiocyanate lysis with rapid purification through a silica-gel membrane. To accommodate multiple applications, the membranes are housed in spin columns of various sizes and in 96-well plates. The RNeasy 96 procedure can be performed manually using a vacuum manifold, a centrifuge, or automated on the BioRobot 9604. To increase RNA yield, from plant tissue, QIAshredder columns are included in the RNeasy Plant Mini Kit. These columns are used for homogenization and filtration of viscous plant and fungal lysates prior to use of the RNeasy spin column.

Roche Molecular Biochemicals' High Pure RNA isolation kits employ a glass-fiber fleece in a spin-filter tube to bind total nucleic acids. Copurified DNA is ultimately digested by a DNase digestion step. Kits are available for isolation of RNA from cultured cells, tissue, and viruses.

The StrataPrep Total RNA Miniprep kit isolates total RNA from a variety of tissues and cells from a wide range of sample quantities. Designed for experiments requiring small amounts of RNA, the protocol includes a specific DNA removal step that makes it ideal for preparing total RNA for RT-PCR. The microspin-cup format allows large numbers of samples to be processed simultaneously.

Magnetic separation offers a rapid means of separating RNA. Superparamagnetic particles, which can be made from a number of substances such as polystyrene or iron oxide and polysaccharides, are magnetic when placed in a magnetic field, but retain no residual magnetism when removed from the magnetic field. This lack of residual magnetism ensures that the particles can be repeatedly separated and resuspended without magnetically induced aggregation.

The RiboMag Total RNA Isolation Kit from Advanced Biotechnologies combines magnetic separation and silica adsorption for the isolation of total RNA. Following a nonphenol lysis step and a quick spin to pellet cell walls, the supernatant is mixed with magnetic silica. For quantities over 10 μg, the magnetic separation can be substituted with alcohol precipitation. Magnetic separators are available for 10 or 20 1.5-ml tubes and 96-well plates. Advanced Biotechnologies also offers a phenol guanidine-based Total RNA Isolation Reagent (TRIR), for single-step isolation of total RNA from tissues, cells, bacteria, plants, yeast, and biological fluids.

However, bearing in mind that total RNA comprises more than just mRNA. the specific analysis of mRNA can not be ideal when it merely forms a smaller component of total RNA—particularly where the particular mRNA transcripts of interest are in very low copy number.

To this end, since it has been recently determined that exosome-derived mRNA may be full length and polyadenylated, this has enabled the development of methods of specifically isolating exosome mRNA based on targeting the poly (A) tail. Methods of targeting and isolating polyadenylated RNA are well known in the art and are easily and routinely applied.

The RNA amplification or probing steps of the present diagnostic invention rely on the use of primers. Reference to a “primer” or an “oligonucleotide primer” should be understood as a reference to any molecule comprising a sequence of nucleotides, or functional derivatives or analogues thereof, the function of which includes hybridisation to a region of a nucleic acid molecule of interest. It should be understood that the primer may comprise non-nucleic acid components. For example, the primer may also comprise a non-nucleic acid tag such as a fluorescent or enzymatic tag or some other non-nucleic acid component which facilitates the use of the molecule as a probe or which otherwise facilitates its detection or immobilisation. The primer may also comprise additional nucleic acid components, such as the oligonucleotide tag which is discussed in more detail hereinafter. In another example, the primer may be a protein nucleic acid which comprises a peptide backbone exhibiting nucleic acid side chains.

The design and synthesis of primers suitable for use in the present invention would be well known to those of skill in the art. In one embodiment, the subject primer is 4 to 60 nucleotides in length, in another embodiment 10 to 50 in length, in yet another embodiment 15 to 45 in length, in still another embodiment 20 to 40 in length, in yet another embodiment 25 to 35 in length. In yet still another embodiment, primer is about 26, 27, 28, 29, 30, 31, 32, 33 or 34 nucleotides in length.

Various techniques can be used to analyse an amplification product in order to determine relative gene expression levels. Their operational characteristics, such as ease of use or sensitivity, vary so that different techniques may be useful for different purposes. They include but are not limited to:
Sequencing
Pyrosequencing
Enzyme digestion
Microarray analysis
Denaturing gradient gel electrophoresis
Agarose gel based separation
Melt curve analysis on real-time PCR cyclers
Quantitative real-time PCR
Denaturing high performance liquid chromatography
Mass spectrometry
Primer extension
Oligonucleotide-ligation
Mutation specific polymerase chain reaction
Denaturing gradient electrophoresis (DGGE)
Temperature gradient denaturing electrophoresis
Constant denaturing electrophoresis
Single strand conformational electrophoresis
Denaturing high performance liquid chromatography (DHPLC)

In terms of the detection of the protein expression product, testing for a proteinaceous expression product in a biological sample can be performed by any one of a number of suitable methods which are well known to those skilled in the art. Examples of suitable methods include, but are not limited to, antibody based screening such as in the context of Western blotting, ELISA, immunohistochemistry or flow cytometry procedures. These, of course, include both single-site and two-site or “sandwich” assays of the non-competitive types, as well as in the traditional competitive binding assays. These assays also include direct binding of a labelled antibody to a target.

It is well within the skill of the person of skill in the art to select and apply an appropriate method of screening for the gene marker expression levels hereinbefore discussed.

The present invention is further described by reference to the following non-limiting examples.

### Example 1

To test the detectability of gene markers in blood plasma specimens, commercially available TaqMan assays were purchased from Applied Biosystems. Positioning of PCR amplicon locations (ie which exon-exon junction) was guided by the Human ST Exon 1.0 microarray study of 42 matched normal specimens and serrated adenomas, ie. Towards exons showing the highest fold difference between normal and adenomous colon specimens.

A total of 68 TaqMan assays targeting a total of 46 genes (red/green coloured genes in Appendix 1 and 2) were used on 2.5 uL cDNA generated from RNA (RNA:cDNA 1:1) extracted from 2 mL plasma. Plasma was produced from two consecutive centrifugation steps (1,500 g, 10 min, 4 deg C) of whole blood collected in 9 mL K3-EDTA vacutainer blood tubes. The TaqMan assays were tested on a least one panel of 45 blood plasma specimens from 15 normal patient, 15 patients with colorectal adenomas and 15 patients with colorectal cancer (phenotypes obtained by colonoscopy). Table 1 (and Tables 2 and 3) summarises the signals of RNA derived from 46 unique genes

It is apparent from Table 2 that the detectability of a tissue mRNA in blood plasma does not correlate with the expression levels observed in colorectal neoplastic tissue specimens. For example, of the top 5 differentially expressed genes in neoplastic colon tissue specimens relative to non-neoplastic controls, three transcripts namely DPEP1, MMP7 and CDH3, were not detectable in human blood plasma obtained from patients with colorectal cancer using even very sensitive methodologies. Conversely, some of those colorectal tissue biomarkers which demonstrated only relatively little differential expression between normal and neoplastic specimens were readily detected in blood plasma specimens, for example CRNDE and OLFM4.

Factors such as amplicon location (i.e. what exon-exon junction is amplified by the PCR assay), amplicon size (larger amplicon size are more difficult to PCR amplify), the number of splice variants amplified by a PCR assays, chromosome strand and chromosomal position, the size of the mRNA and subcellular location of mRNAs were investigated. No correlation was seen between these factors relative to the detectability of the mRNA in blood plasma.

However, an unexpected and surprising correlation that was observed was the correlation between the plasma detectability of some mRNA targets with presumed exosomal contents. This led to the determination that some gene markers which are increased in expression in colorectal neoplasia are actually detectable at significantly increased levels in exosomes, while others (surprisingly) are not.

### Example 2

### Materials and Methods

### Clinical Specimens

Blood specimens from healthy donors (136), adenoma (124, any grade), and cancer (138, any grade) patients were procured through collaboration with Flinders Medical Center (Adelaide, Australia) or from a clinical specimen vendor (Proteogenex, USA). Colorectal neoplastic status was confirmed by colonoscopy and pathology review for all specimens. Plasma was generated from whole blood phlebotomy specimens (K3EDTA Vacutainer) within 4 hrs of blood draw using a 2×1,500 g spin protocol.

Plasma RNA Extraction, Generation of cDNA Libraries and Extraction Quality Control

RNA was extracted from 2 mL plasma aliquots using the QIAamp Circulating Nucleic Acid Extraction Kit (Qiagen, Australia) and eluted into a final volume of 100pL. To normalize for nucleic acid extraction efficiency differences between specimens, arRNA enterovirus (Asuragen, US) was spiked into each plasma specimen prior to RNA isolation and recovery was measured downstream of the extraction procedure. Ten distinct cDNA panels (each containing 15 healthy donors, 15 adenomas and 15 cancers) were generated by converting 10 μL RNA into 20 μL cDNA reactions (SuperScript®, Invitrogen, USA).

Plasma mRNA Expression Analysis by qRT-PCR

Tissue to plasma expression portability was examined using Taqman Gene Expression Assays (Applied Biosystems, USA) targeting 48 unique genes. Assays were run in triplicate on 2.5 μL cDNA per patient. Patient fold changes were calculated as patient mean Ct values (arRNA corrected) relative to the median Ct value for ‘normal’ specimens.

Analysis of Plasma-Detectable Vs. Non-Detectable mRNA Transcripts

The subset of mRNA detectable in plasma was compared to mRNA transcripts that were not detectable in plasma across a range of descriptor covariates (e.g. amplicon length, chromosomal location, GC-content, etc.). In particular, the correspondence of mRNA expression to the evidence that particular mRNAs or proteins have been observed to be detectable in exosomes derived from a range of human and murine tissue cells were assessed.

### Results

(1) Total RNA is Readily Extracted from Plasma Specimens

GAPDH was detected in all the 398 blood plasma specimens analyzed (Ct mean 30.21; 95% CI: 27.5 to 32.9) using the commercially available TaqMAN Gene Expression GAPDH Assay Hs99999905_ml (Applied Biosystems, USA).

### (2) Correlation Between Detectability in Plasma and Biomarker Expression in Colon Tissues

Of the 46 different genes tested, 21 showed no detectable RNA signal in any of the plasma specimens while 22 were detectable but not differentially expressed between cancer and non-neoplastic control plasma. Only 3 mRNA biomarkers validated in tissue were likewise expressed at higher concentration in neoplastic plasma relative to non-neoplastic controls.

(3) KIAA1199 mRNA Levels in Plasma Elevated Neoplastic Specimens

A commercially available TaqMAN Gene Expression KIAA 1199 Assay, Hs01552116_ml, was used to detect KIAA1199 in six cDNA plasma libraries comprising a total of 96 healthy donors and 95 colorectal adenoma and 99 cancer patients with an average sensitivity of 74% (CI: 58-90%) and specificity of 66% (CI: 45-87%) when applying the criteria of designating a sample positive if 2 out of 3 of the triplicates gave a positive qRT-PCR signal.

(4) Detectability of Tissue Biomarkers Correlates with Appearance in Microvesicles

A range of factors were investigated but did not explain the lack of tissue-to-blood presence correspondence, e.g. amplicon size/location, % GC, number of splice variants amplified, transcript size, etc. However, a correspondence between RNA detection in plasma and evidence of exosomal expression was identified. Although this did not fully correlate with the information provided in the ExoCarta database, being a database of exosomal proteins and RNA. Specifically, approximately 30% of the genes with no PCR-amplifiable signal in plasma were nevertheless listed in the ExoCarta database while approximately 30% of the genes which were detectable in plasma did not appear in this database. This may be explained by the fact that this database has been generated from non-human cell line data and demonstrates the unpredictable nature of these results and the fact that the utility of this database must be treated with caution.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
Signals obtained in blood plasma based on
using commercially available TaqMan assays

| | | Signals in blood | Signals in blood |
|---|---|---|---|
| | No signals in | plasma but no | plasma and |
| | blood plasma | phenotypic differences | phenotypic differences |
| # Genes | 21 | 22 | 3 |
| Gene IDs | Table 2 (genes | Table 2 | OLFM4, KIAA1199, |
| | in bold text) | (underlined genes) | CRNDE |

TABLE 2
Up-regulated gene markers (Validated and >2fold up regulated)

| GENE ID | FoldChange | GENE ID | FoldChange | GENE ID | FoldChange |
|---|---|---|---|---|---|
| MMP7 | 69.2940889 | TUBB6 | 5.900896434 | RNF43 | 3.235950018 |
| CDH3 | 37.54418585 | GDF15 | 5.837723023 | COL12A1 | 3.159411751 |
| KIAA1199 | 25.15771962 | ESM1 | 5.770701347 | IL8 | 3.099634128 |
| DPEP1 | 23.90265692 | SPINK4 | 5.532287396 | PUS7 | 3.055262871 |
| TESC | 16.8359447 | PSAT1 | 5.522261448 | PVT1 | 3.032897709 |
| TACSTD2 | 15.71389259 | GALNT6 | 5.273892946 | TIMP1 | 3.01157947 |
| MMP3 | 15.62573272 | PHLDA1 | 5.266112409 | SOX4 | 2.885880403 |
| TCN1 | 15.22244133 | HPGD | 5.163051582 | C20orf199 | 2.86516777 |
| LCN2 | 14.5998521 | SLC6A6 | 5.024940341 | CCND1 | 2.850006054 |
| WDR72 | 14.11279645 | CLCA4 | 4.936369867 | UBE2C | 2.821499493 |
| REG1B | 13.1524883 | SLCO4A1 | 4.839723453 | BGN | 2.688136526 |
| CLDN1 | 13.09118948 | DUSP27 | 4.808410411 | ANLN | 2.629414188 |
| CST1 | 13.0112388 | NFE2L3 | 4.573155915 | ITGA6 | 2.622725944 |
| REG3A | 12.09396998 | MMP11 | 4.548834746 | NQO1 | 2.615683201 |
| TRIM29 | 11.01384395 | CXCL5 | 4.479689229 | GPR56 | 2.601986204 |
| FOXQ1 | 10.96687218 | AXIN2 | 4.438003402 | COL11A1 | 2.601582944 |
| REG1A | 10.93384951 | S100P | 4.371856797 | CTHRC1 | 2.570488121 |
| MMP1 | 10.58656426 | SORD | 4.363772545 | KCNQ1 | 2.531742623 |
| CXCL1 | 10.40161869 | TGFBI | 4.312124487 | COL1A1 | 2.499187467 |
| SERPINB5 | 10.35507344 | SCD | 4.284540732 | IFITM2 | 2.377199791 |
| CCL20 | 10.13514886 | CDCA7 | 4.270186463 | PLCB4 | 2.369558592 |
| RPESP | 9.074780109 | HIG2 | 4.252714357 | FAP | 2.326011697 |
| LGR5 | 7.892049455 | RPL22L1 | 4.185452765 | EXO1 | 2.308015816 |
| CXCL3 | 7.852054187 | CKS2 | 4.153736931 | FERMT1 | 2.280865345 |
| CXCL2 | 7.852054187 | MYC | 4.114034116 | CSE1L | 2.28086168 |
| REG4 | 7.746359355 | MTHFD1L | 4.038731263 | CDH11 | 2.248504671 |
| ASCL2 | 7.733047831 | ENC1 | 3.929471365 | TMEPAI | 2.195453935 |
| DEFA6 | 7.106277407 | IFITM1 | 3.866248082 | LDHB | 2.176297011 |
| KRT23 | 6.809993042 | SOX9 | 3.846462925 | MUC12 | 2.126657261 |
| LY6G6D | 6.64089123 | S100A11 | 3.832630399 | SLC7A1 | 2.073994601 |
| UBD | 6.585360578 | INHBA | 3.758881226 | TPX2 | 2.028679461 |
| CADPS | 6.45432004 | GIF | 3.666184963 | TMEM97 | 2.773664788 |
| TDGF1 | 6.33018716 | SLC12A2 | 3.419383434 | GMDS | 2.699668005 |
| SLC7A5 | 6.308424061 | MET | 3.346688346 | SQLE | 2.692691797 |
| MMP12 | 6.240660755 | ANXA3 | 3.337731608 | NEBL | 5.515804992 |
| AZGP1 | 6.187044505 | SPP1 | 3.324938278 | ECT2 | 2.816640259 |

TABLE 3
Other markers screened in plasma

| | | | |
|---|---|---|---|
| GIF | GPA33 | RPL14 | GAPDH |
| HTERT | AOF2 | SDHA | OLFM4 |
| JUB | AUTS2 | CRNDE | |
| SLITRK6 | ACTB | ALDOA | |

TABLE 4
Detectability of mRNA transcripts in human plasma

| | Amplicon detected | Amplicon detected |
|---|---|---|
| No Signal in plasma | NO phenotypic profile | Phenotypic profile |
| 21 out of 46 tested (46%) | 22 out of 46 tested (48%) | 3 out of 46 tested (6%) |
| ASCL2; CDH3; COL11A1; | AOF2; AUTS2; CXCL3; | CRNDE; KIAA1199; |
| DEFA6; GDF15; GIF; hTERT; | GALNT6; TESC; SNORD12; | OLFM4 |
| INHBA; JUB; LGR5; MET; | ACTB; ALDOA; GAPDH; GMDS; | |
| RNF43; SLITRK6; TCN1; | IFITM1; ITGA6; LCN2; PUS7; | |
| NFE2L3; DPEP1; FOXQ1; | RPL14; S100A11; SLC12A2; | |
| GPA33; MMP7; NEBL; REG4 | SORD; TGFBI; S100P; SDHA; | |

TABLE 5

| Gene Name | Chromosomal Co-ordinates (Hg19) |
|---|---|
| KIAA1199 | chr15: 81,071,712-81,243,999 |
| CRNDE | chr16: 54,952,778-54,963,079 |
| OLFM4 | chr13: 53,602,876-53,626,196 |
| DPEP1 | chr16: 89,687,000-89,704,839 |
| TESC | chr12: 117,476,728-117,537,251 |
| SLC12A2 | chr5: 127,419,483-127,525,380 |
| ITGA6 | chr2: 173,292,314-173,371,181 |
| REG4 | chr1: 120,336,641-120,354,203 |
| S100A11 | chr1: 152,004,982-152,009,511 |
| ACAA2 | chr18: 47,309,874-47,340,251 |
| ANPEP | chr15: 90,328,126-90,358,072 |
| ANXA3 | chr4: 79,472,742-79,531,605 |
| APP | chr21: 27,252,861-27,543,138 |
| APPL2 | chr12: 105,567,075-105,630,008 |
| AZGP1 | chr7: 99,564,350-99,573,735 |
| BGN | chrX: 152,760,347-152,775,004 |
| c20orf199 | chr20: 47894715-47905797, chr20: 47894715-47905797, |
| | chr20: 47894715-47905797, chr20: 47894715-47905797, |
| | chr20: 47894808-47905797, chr20: 47895179-47905797, |
| | chr20: 47895179-47905797 |
| CALR | chr19: 13,049,414-13,055,304 |
| CAP1 | chr1: 40,506,255-40,538,321 |
| COL12A1 | chr6: 75,794,042-75,915,623 |
| CSE1L | chr20: 47,662,838-47,713,486 |
| CTSC | chr11: 88,026,760-88,070,941 |
| CXCL3 | chr4: 74,902,312-74,904,490 |
| DMBT1 | chr10: 124,320,181-124,403,252 |
| ENO1 | chr1: 8,921,059-8,939,151 |
| EPS8L3 | chr1: 110,292,702-110,306,644 |
| FAT FAT4 | chr4: 126315091-126414087 |
| FAT2 | chr5: 150935821-150948505 |
| FAT1 | chr4: 187627717-187647850 |
| FAT1 | chr4: 187508937-187516980 |
| FAT2 | chr5: 150883653-150948505 |
| FAT2 | chr5: 150883653-150911531 |
| FAT1 | chr4: 187508937-187644987 |
| FAT4 | chr4: 126237567-126414087 |
| FAT4 | chr4: 126369616-126412943 |
| FAT3 | chr11: 92085262-9262963 5 |
| FAT3 | chr11: 92573728-92629635 |
| FTH1 | chr11: 61,731,757-61,735,132 |
| GALNT6 | chr12: 51,745,833-51,785,200 |
| GMDS | chr6: 1,624,035-2,245,868 |
| GNB2L1 | chr5: 180,663,928-180,670,906 |
| GPRC5A | chr12: 13,043,956-13,066,600 |
| HEPH | chrX: 65,382,433-65,487,230 |
| HLADRB1 | chr6 : 3,774,425-3,787,546 |
| HPGD | chr4: 175,411,328-175,443,792 |
| HSP9OAA1 | chr14: 102547075-102606086 |
| IFITM1 | chr11: 313,991-315,272 |
| IFITM2 | chr11: 308,107-309,410 |
| KRT8 | chr12: 53,290,971-53,298,868 |
| LCN2 | chr9: 130,911,732-130,915,734 |
| LDHB | chr12: 21,788,275-21,810,789 |
| LIMA1 | chr12: 50,569,563-50,677,353 |
| LOC440264 | chr15: 30,262,050-30,265,947 |
| LRPPRC | chr2: 44,113,363-44,223,144 |
| LRSAM1 | chr9: 130,214,534-130,265,780 |
| MLLT3 | chr9: 20,344,968-20,622,514 |
| MMP7 | chr11: 102,391,239-102,401,478 |
| MUC13 | chr3: 124,624,289-124,653,595 |
| MYO5B | chr18: 47,349,156-47,721,451 |
| NDRG1 | chr8: 134,249,414-134,309,547 |
| NEBL | chr10: 21,068,903-21,186,531 |
| NQO1 | chr16: 69,743,304-69,760,533 |
| OLA1 | chr2: 174,937,175-175,113,365 |
| PIGR | chr1: 207,101,867-207,119,811 |
| PRDX1 | chr1: 45,976,707-45,988,562 |
| PROS1 | chr3: 93,591,881-93,692,934 |
| PSAT1 | chr9: 80,912,059-80,945,009 |
| PUS7 | chr7: 105,096,960-105,162,685 |
| RAB8A | chr19: 16,222,490-16,244,445 |
| RPL6 | chr12: 112,842,994-112,847,443 |
| RPS4X | chrX: 71,492,453-71,497,141 |
| RPS7 | chr2: 3,622,853-3,628,509 |
| S100A1 | chr1: 153,600,873-153,604,513 |
| S100A6 | chr1: 153,507,076-153,508,717 |
| S100P | chr4: 6,695,566-6,698,897 |
| SLC39A5 | chr12: 56,623,820-56,631,629 |
| SLC7A5 | chr16: 87,863,629-87,903,100 |
| SLK | chr10: 105,727,470-105,787,342 |
| SOD1 | chr21: 33,031,935-33,041,243 |
| SORD | chr15: 45,315,302-45,367,287 |
| TACSTD2 | chr1: 59,041,095-59,043,166 |
| TCP1 | chr6: 160,199,530-160,210,735 |
| TFRC | chr3: 195,776,155-195,809,032 |
| TGFBI | chr5: 135,364,584-135,399,507 |
| THBS2 | chr6: 169,615,875-169,654,137 |
| TM7SF3 | chr12: 27,124,506-27,167,339 |
| TUBB6 | chr18: 12,308,257-12,326,568 |
| VAMP3 | chr1: 7,831,329-7,841,492 |
| VAT1 | chr17: 41,166,622-41,174,459 |

### BIBLIOGRAPHY

Chirgwin et al. Biochemistry, 18(24):5294-9, 1979)
Chomczynski and Sacchi (Analytical Biochemistry, 162(1):156-9, 1987

1. A method of screening for the onset or a predisposition to the onset of a large intestine neoplasm in an individual, said method comprising measuring the level of expression of:
(i) any one or more genes selected from:

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 |
| 9. S100A11 | 10. ACAA2 | 11. ANPEP | 12. ANXA3 |
| 13. APP | 14. APPL2 | 15. AZGP1 | 16. BGN |
| 17. c20orf199 | 18. CALR | 19. CAP1 | 20. COL12A1 |
| 21. CSE1L | 22. CTSC | 23. CXCL3 | 24. DMBT1 |
| 25. ENO1 | 26. EPS8L3 | 27. FAT | 28. FTH1 |
| 29. GALNT6 | 30. GMDS | 31. GNB2L1 | 32. GPRC5A |
| 33. HEPH | 34. HLADRB1 | 35. HPGD | 36. HSP9OAA1 |
| 37. IFITM1 | 38. IFITM2 | 39. KRT8 | 40. LCN2 |
| 41. LDHB | 42. LIMA1 | 43. LOC440264 | 44. LRPPRC |
| 45. LRSAM1 | 46. MLLT3 | 47. MMP7 | 48. MUC13 |
| 49. MYO5B | 50. NDRG1 | 51. NEBL | 52. NQO1 |
| 53. OLA1 | 54. PIGR | 55. PRDX1 | 56. PROS1 |
| 57. PSAT1 | 58. PUS7 | 59. RAB8A | 60. RPL6 |
| 61. RPS4X | 62. RPS7 | 63. S100A1 | 64. S100A6 |
| 65. S100P | 66. SLC39A5 | 67. SLC7A5 | 68. SLK |
| 69. SOD1 | 70. SORD | 71. TACSTD2 | 72. TCP1 |
| 73. TFRC | 74. TGFBI | 75. THBS2 | 76. TM7SF3 |
| 77. TUBB6 | 78. VAMP3 or | 79. VAT1; | |

or
(ii) any one of more of the regions defined by Hg19 coordinates:

| | |
|---|---|
| 1. chr15: 81,071,712-81,243,999 | 2. chr16: 54,952,778-54,963,079 |
| 3. chr13: 53,602,876-53,626,196 | 4. chr16: 89,687,000-89,704,839 |
| 5. chr12: 117,476,728-117,537,251 | 6. chr5: 127,419,483-127,525,380 |
| 7. chr2: 173,292,314-173,371,181 | 8. chr1: 120,336,641-120,354,203 |
| 9. chr1: 152,004,982-152,009,511 | 10. chr18: 47,309,874-47,340,251 |
| 11. chr15: 90,328,126-90,358,072 | 12. chr4: 79,472,742-79,531,605 |
| 13. chr21: 27,252,861-27,543,138 | 14. chr12: 105,567,075-105,630,008 |
| 15. chr7: 99,564,350-99,573,735 | 16. chrX: 152,760,347-152,775,004 |
| 17. chr20: 47894715-47905797 | 18. chr20: 47894715-47905797 |
| 19. chr20: 47894715-47905797 | 20. chr20: 47894715-47905797 |
| 21. chr20: 47894808-47905797 | 22. chr20: 47895179-47905797 |
| 23. chr20: 47895179-47905797 | 24. chr19: 13,049,414-13,055,304 |
| 25. chr1: 40,506,255-40,538,321 | 26. chr6: 75,794,042-75,915,623 |
| 27. chr20: 47,662,838-47,713,486 | 28. chr11: 88,026,760-88,070,941 |
| 29. chr4: 74,902,312-74,904,490 | 30. chr10: 124,320,181-124,403,252 |
| 31. chr1: 8,921,059-8,939,151 | 32. chr1: 110,292,702-110,306,644 |
| 33. chr4: 126315091-126414087 | 34. chr5: 150935821-150948505 |
| 35. chr4: 187627717-187647850 | 36. chr4: 187508937-187516980 |
| 37. chr5: 150883653-150948505 | 38. chr5: 150883653-150911531 |
| 39. chr4: 187508937-187644987 | 40. chr4: 126237567-126414087 |
| 41. chr4: 126369616-126412943 | 42. chr11: 92085262-92629635 |
| 43. chr11: 92573728-92629635 | 44. chr11: 61,731,757-61,735,132 |
| 45. chr12: 51,745,833-51,785,200 | 46. chr6: 1,624,035-2,245,868 |
| 47. chr5: 180,663,928-180,670,906 | 48. chr12: 13,043,956-13,066,600 |
| 49. chrX: 65,382,433-65,487,230 | 50. chr6 : 3,774,425-3,787,546 |
| 51. chr4: 175,411,328-175,443,792 | 52. chr14: 102547075-102606086 |
| 53. chr11: 313,991-315,272 | 54. chr11: 308,107-309,410 |
| 55. chr12: 53,290,971-53,298,868 | 56. chr9: 130,911,732-130,915,734 |
| 57. chr12: 21,788,275-21,810,789 | 58. chr12: 50,569,563-50,677,353 |
| 59. chr15: 30,262,050-30,265,947 | 60. chr2: 44,113,363-44,223,144 |
| 61. chr9: 130,214,534-130,265,780 | 62. chr9: 20,344,968-20,622,514 |
| 63. chr11: 102,391,239-102,401,478 | 64. chr3: 124,624,289-124,653,595 |
| 65. chr18: 47,349,156-47,721,451 | 66. chr8: 134,249,414-134,309,547 |
| 67. chr10: 21,068,903-21,186,531 | 68. chr16: 69,743,304-69,760,533 |
| 69. chr2: 174,937,175-175,113,365 | 70. chr1: 207,101,867-207,119,811 |
| 71. chr1: 45,976,707-45,988,562 | 72. chr3: 93,591,881-93,692,934 |
| 73. chr9: 80,912,059-80,945,009 | 74. chr7: 105,096,960-105,162,685 |
| 75. chr19: 16,222,490-16,244,445 | 76. chr12: 112,842,994-112,847,443 |
| 77. chrX: 71,492,453-71,497,141 | 78. chr2: 3,622,853-3,628,509 |
| 79. chr1: 153,600,873-153,604,513 | 80. chr1: 153,507,076-153,508,717 |
| 81. chr4: 6,695,566-6,698,897 | 82. chr12: 56,623,820-56,631,629 |
| 83. chr16: 87,863,629-87,903,100 | 84. chr10: 105,727,470-105,787,342 |
| 85. chr21: 33,031,935-33,041,243 | 86. chr15: 45,315,302-45,367,287 |
| 87. chr1: 59,041,095-59,043,166 | 88. chr6: 160,199,530-160,210,735 |
| 89. chr3: 195,776,155-195,809,032 | 90. chr5: 135,364,584-135,399,507 |
| 91. chr6: 169,615,875-169,654,137 | 92. chr12: 27,124,506-27,167,339 |
| 93. chr18: 12,308,257-12,326,568 | 94. chr1: 7,831,329-7,841,492 or |
| 95. chr17: 41,166,622-41,174,459 | |

in a membranous microvesicle sample from said individual wherein an increase in the level of expression of any one or more of said genes or any one of more of said regions defined by Hg19 coordinates relative to a control level of expression is indicative of the onset or the predisposition to the onset of a large intestine neoplasm in said individual.

2-13. (canceled)

14. The method according to claim 1 wherein said large intestine neoplasm is an adenoma.

15. The method according to claim 1 wherein said large intestine neoplasm is an adenocarcinoma.

16. The method according to claim 1 wherein said large intestine neoplasm is a colorectal neoplasm.

17. The method according to claim 1 wherein either the level of RNA transcribed of any one or more of said genes or any one of more of said regions defined by Hg19 coordinates or cDNA reverse transcribed therefrom is measured.

18. The method according to claim 17 wherein said RNA is mRNA.

19. The method according to claim 1 wherein the level of a protein expression product or fragment thereof of said any one or more of said genes or any one of more of said regions defined by Hg19 coordinates is measured.

20. The method according to claim 1 wherein said microvesicle is an exosome, apoptotic bleb, microparticle, apoptotic body or a cellular bleb.

21. The method according to claim 1 wherein said microvesicle is an exosome.

22. The method according to claim 1 wherein said biological sample is blood, serum, plasma, urine, stool, saliva, tears or ascites fluid.

23. The method according to claim 1 wherein any one or more of the following genes are measured:
(i)

| | | | |
|---|---|---|---|
| 1. KIAA1199 | 2. CRNDE | 3. OLFM4 | 4. DPEP1 |
| 5. TESC | 6. SLC12A2 | 7. ITGA6 | 8. REG4 or |
| 9. S100A11; | | | |

or
(ii) any one or more of the following regions defined by Hg19 coordinates are measured:

| | |
|---|---|
| 1. chr15: 81,071,712-81,243,999 | 2. chr16: 54,952,778-54,963,079 |
| 3. chr13: 53,602,876-53,626,196 | 4. chr16: 89,687,000-89,704,839 |
| 5. chr12: 117,476,728-117,537,251 | 6. chr5: 127,419,483-127,525,380 |
| 7. chr2: 173,292,314-173,371,181 | 8. chr1: 120,336,641-120,354,203 or |
| 9. chr1: 152,004,982-152,009,511. | |

24. The method according to claim 23 wherein any one or more of the following genes are measured:
(i)

| | | |
|---|---|---|
| 1. KIAA1199 | 2. CRNDE or | 3. OLFM4; |

or
(ii) any one or more of the following regions defined by Hg19 coordinates are measured:

| | |
|---|---|
| 1. chr15: 81,071,712-81,243,999 | 2. chr16: 54,952,778-54,963,079 or |
| 3. chr13: 53,602,876-53,626,196. | |

25. The method according claim 1 wherein said individual is a human.
